(19) United States
(12) Patent Application Publication
(10) Pub. No.: US 2022/0211063 A1
(43) Pub. Date: Jul. 7, 2022

(54) FUNGAL STRAINS, PRODUCTION AND USES THEREOF

(71) Applicant: UNIVERSITY OF NOTTINGHAM, Nottingham (GB)

(72) Inventor: Paul DYER, Nottingham (GB)

(73) Assignee: UNIVERSITY OF NOTTINGHAM, Nottingham (GB)

(21) Appl. No.: 17/604,485

(22) PCT Filed: Apr. 16, 2020
(86) PCT No.: PCT/EP2020/060793

§ 371 Date: Oct. 18, 2021

(30) Foreign Application Priority Data
Apr. 16, 2019 (GB) 1905360.2

(51) Int. Cl. A23C 19/032; C12N 3/00; C12N 1/14; A23C 19/068; G01N 30/72

(52) U.S. Cl. CPC A23C 19/032; C12N 3/00; C12N 1/14; C12R 2001/80; A23C 19/0682; G01N 30/7206; C12N 1/145

(57) ABSTRACT

This invention relates to novel Penicillium strains, the production of novel Penicillium strains, and the use of novel Penicillium strains in various end applications.

### TECHNICAL FIELD

This invention relates to novel Penicillium strains, the production of novel Penicillium strains, and the use of novel Penicillium strains in various applications.

### BACKGROUND

Penicillium species are generally ubiquitous, opportunistic saprophytic fungi, being found on a variety of decaying organic matter. They have been isolated from various substrates and environments including air, soil, various food and feed products and indoor environments. They include species that are vitally important in the medical, biotechnological and food industries.

The name Penicillium is derived from Latin (penicillus: paintbrush) and was first described in 1809. Species in this genus are characterised by the production of an asexual reproductive structure known as a penicillus. Most of the species in this genus produce green or blue-green colonies of branched, septate conidiophores, with sporulating cells (phialides or sterigmata) clustered at the ends on which the conidia are located; this structure is known as a penicillus. Most Penicillium species produce conidia of similar colour.

A number of strains of Penicillium species are used in the production of mould-ripened cheese worldwide, for example P. roqueforti and P. camemberti are used in the production of Roquefort and Camembert cheeses, respectively. Blue cheeses, such as Roquefort, are by definition cheeses that are ripened primarily by the growth and activity of appropriate strains of P. roqueforti throughout the cheese mass rather than on the surface only. Whereas in other cheeses, such as Camembert, the fungal growth, in this case P. camemberti, is on the cheese surface.

Until recently, the majority of Penicillium species were only known as asexual organisms, meaning that it was very difficult to generate novel genetic diversity to make different strains. As a result, relatively few strains were available for cheese production and other applications. Strain improvement therefore mainly relies on random mutagenesis, followed by screening for variants exhibiting enhanced properties of interest such as growth, mycelium thickness and colony colour or lipolytic and proteolytic actions.

As described herein, an improved method for making sexual crosses of Penicillium species, and Penicillium roqueforti in particular, means that new strains with new properties, including new flavours, can be more readily made. Other desirable properties can also be selected for in the sexual crosses, including commercial traits such as faster growth and increased shelf life.

Creating genetic variation through sexual crossing furthermore enables the targeted development of novel strains that provide a benefit to the product manufacturer and/or the consumer in terms of quality and sensorial factors.

### SUMMARY OF THE INVENTION

The inventors have developed an improved method for sexually reproducing Penicillium. The improved method improves the chance of mating, which in turn makes the method for production more economically efficient than conventional methods for Penicillium strain production.

The inventors have also developed novel strains of Penicillium and novel Penicillium derivatives having changed spore colour. The inventors have also isolated and characterised novel strains of Penicillium from silage located in the West Midlands, England, United Kingdom.

A first aspect of the present invention provides novel strains of Penicillium selected from:
(a) strain Penicillium roqueforti 5A (74-256) deposited under the Budapest Treaty with accession no. CBS 145358, referred to herein as “5A”;
(b) strain Penicillium roqueforti 12A (74-257) deposited under the Budapest Treaty with accession no. CBS 145359, referred to herein as “12A”;
(c) strain Penicillium roqueforti A22 (74-160) deposited under the Budapest Treaty with accession no. CBS 145360, referred to herein as “A22”;
(d) strain Penicillium roqueforti B20 (74-170) deposited under the Budapest Treaty with accession no. CBS 145361, referred to herein as “B20”;
(e) a strain obtainable by sexually crossing strains of Penicillium roqueforti 74-130 and 74-144 and having similar characteristics to A22;
(f) a strain obtainable by sexually crossing strains of Penicillium roqueforti 74-133 and 74-146 and having similar characteristics to B20;
(g) variant strains having at least 99.0% sequence identity to the genome sequence of any of the strains defined in (a) to (f).

Strains 5A (CBS 145358), 12A (CBS 145359), A22 (CBS 145360) and B20 (CBS 145361) were deposited under the Budapest Treaty at the Westerdijk Fungal Biodiversity Institute (an International Depositary Authority registered under the Budapest Treaty), Utrecht, The Netherlands, on 24 Jan. 2019. The deposit was made by Dr Paul S. Dyer of the University of Nottingham, School of Life Sciences, University Park, Nottingham NG7 2RD, United Kingdom.

Penicillium roqueforti strains 74-130, 74-144, 74-133, 74-146 may be found at the Nottingham University BDUN Collection.

The novel strains exhibit certain characteristics which differ from one or both parental strains used in the sexual cross, or relative to known strains used in the manufacture of dairy products. A person skilled in the art would readily be aware of the known and commercially-available Penicillium strains typically used in the manufacture of dairy products.

For example, the novel strains may exhibit altered protease and/or lipase levels and/or activity. Additionally or alternatively, the novel strains may have altered levels of certain secondary metabolites. Additionally or alternatively, when used in the production of dairy products, the novel strains may alter the organoleptic profile of the dairy product. In each case, the altered characteristic is altered either compared to one or both parental strains used in the sexual cross, or relative to known strains used in the manufacture of dairy products.

The parental strains used in a sexual cross may be any known strain of Penicillium, optionally any known strain of Penicillium roqueforti. In one embodiment, the parental strain may be selected from at least one of: Penicillium roqueforti 74-130, 74-144, 74-133, 74-146, 74-88, 74-92. The aforementioned numbers refer to strains which may be found at the Nottingham University BDUN Collection.

Strain A22 may be derived from a cross of parental strains of Penicillium roqueforti 74-130 and 74-144. Strain B20 may be derived from a cross of parental strains Penicillium roqueforti 74-133 and 74-146.

In order to generate further novel strains, any known Penicillium strain may be crossed with one of the novel strains described under any of (a) to (g) above, such as strains 5A, 12A, A22, B20 and variants thereof. Optionally, the novel strains listed under (a) to (g) above may be crossed with each other.

Reference herein to an “organoleptic profile” refers to sensory features of the dairy product in question, particularly cheese, such as the appearance, smell, flavour, texture, mouthfeel or any such attribute which contributes to eating quality.

The organoleptic profile may, for example, be a flavour profile. The flavour profile conferred by the Penicillium strains to any given food product may be determined using any known method. For example, methods such as principal component analysis of flavour volatiles (such as methyl ketones, alcohols etc.) produced on, for example, milk media as detected by GC-MS methodology, may be used to determine a flavour profile (see FIG. 10 for one such example).

According to one embodiment, the invention provides Penicillium strains having a flavour profile that is substantially similar to the flavour profile of strains 5A, 12A, A22, B20, for example as shown in FIG. 10. Whether a strain has a “substantially similar” flavour profile may readily be determined by a skilled person using any suitable technique, such as GC-MS. A strain having a substantially similar flavour profile to any of strains 5A, 12A, A22, B20 may also share other similarities, such as enzyme activity and levels and/or levels of certain secondary metabolites.

The altered levels of any given mycotoxin (such as such as mycophenolic acid, Roquefortines (A, B and C), PR toxin and Penillic acid) found in the novel strains of the invention may be lower or higher compared to levels of the corresponding mycotoxin produced by known strains of Penicillium roqueforti, such as known strains used in cheese production. Strains with altered levels of mycotoxin production relative to current production strains may be beneficial for cheese production, with some mycotoxins even having health promoting benefits, making increased levels of certain mycotoxins beneficial. The presence and levels of mycotoxins may be detected and measured using any known technique, such as liquid chromatographic techniques (e.g. HPLC).

Variants of strains 5A, 12A, A22 and B20 include strains having at least 99.0%, 99.1%, 99.2%, 99.3%, 99.4%, 99.5%, 99.6%, 99.7%, 99.8%, 99.9% or more sequence identity to the genomic DNA sequence of strains 5A, 12A, A22 and B20. The genomic sequence for the aforementioned strains may be represented by any of the SEQ ID NOs listed below or sequences having at least 99.0%, 99.1%, 99.2%, 99.3%, 99.4%, 99.5%, 99.6%, 99.7%, 99.8%, 99.9% or more sequence identity thereto.

Strain 5A: the supercontigs represented by SEQ ID NO: 7 to SEQ ID NO: 54 or sequences having at least 99.0% sequence identity to any one or more of the aforementioned SEQ ID NOs;

Strain 12A: the supercontigs represented by SEQ ID NO: 55 to SEQ ID NO: 102 or sequences having at least 99.0% sequence identity to any one or more of the aforementioned SEQ ID NOs;

A22: the supercontigs represented by SEQ ID NO: 103 to SEQ ID NO: 150 or sequences having at least 99.0% sequence identity to any one or more of the aforementioned SEQ ID NOs;

B20: the supercontigs represented by SEQ ID NO: 151 to SEQ ID NO: 198 or sequences having at least 99.0% sequence identity to any one or more of the aforementioned SEQ ID NOs.

A person skilled in the art would readily appreciate how to compile the sequence of a genome based on the contigs/supercontigs provided herein.

The at least 99.0% sequence identity may be with reference to the SEQ ID NOs mentioned above or to the genomic sequence of the strain as deposited under the Budapest Treaty at the Westerdijk Fungal Biodiversity Institute: CBS 145358 (5A), CBS 145359 (12A), CBS 145360 (A22) and CBS 145361 (B20).

Identity in this context may be determined using the TBLASTN computer program with the DNA genome sequence for strains 5A, 12A, A22, B20. The BLAST software is publicly available at http://blast.ncbi.nlm.nih.gov/Blast.cgi (accessible on 12 Mar. 2009). The variant strains suitably also exhibit the same or similar biological and functional characteristics as the unmodified strain from which they are derived or on which they are based. The variant strain may have a different or similar organoleptic, for example, flavour profile to strain 5A, 12A, A22 and B20. The variant strain may have similar or different activities or levels of various enzymes and/or levels of certain secondary metabolites.

The abovementioned characteristics listed under any one or more of (a) to (g) do not appear in analyses of other available Penicillium strains. Further differences between strains 5A, 12A, A22 and B20 are provided in the tables below, which differences are relative to a known reference genome, ‘FM164’ (see: https://funqi.ensembl.orq/Penicillium roqueforti fm164 qca 000513255/Info/Index and https://www.ebi.ac.uk/ena/data/view/GCA 000513255.1) and Cheeseman et al., 2014: Multiple recent horizontal transfers of a large genomic region in cheese making fungi (Nature Communications 5: 2876. DOI: 10.1038/ncomms3876). The differences not only distinguish the strains from the reference genome, but also from each other. Although not exhaustive, one or more of the differences shown in the tables below may be used as a diagnostic to help identify the strains of the invention.

TABLE A
Differences between reference genome and strain 5A

| Contig | Position | Ref | Alt | Type | Rank |
|---|---|---|---|---|---|
| HG792015.1 | 6999870 | C | CCAA | disruptive_inframe_insertion | MODERAIE |
| HG792015.1 | 6999882 | C | CCAA | disruptive_inframe_insertion | MODERATE |
| HG792016.1 | 109210 | G | A | missense_variant | MODERATE |
| HG792016.1 | 109283 | C | T | synonymous_variant | LOW |
| HG792016.1 | 109294 | C | T | missense_variant | MODERATE |
| HG792016.1 | 109295 | G | A | synonymous_variant | LOW |
| HG792016.1 | 109300 | G | A | missense_variant | MODERATE |
| HG792016.1 | 109302 | G | A | missense_variant | MODERATE |
| HG792016.1 | 109312 | TG | T | frameshift_variant | HIGH |
| HG792016.1 | 109316 | G | GGTAT | frameshift_variant | HIGH |
| HG792016.1 | 109318 | ACCG | A | stop_gained&disruptive_inframe_deletion | HIGH |
| HG792016.1 | 109324 | G | A | missense_variant | MODERATE |
| HG792016.1 | 109329 | G | A | missense_variant | MODERATE |
| HG792016.1 | 109336 | C | T | missense_variant | MODERATE |
| HG792016.1 | 109339 | C | T | missense_variant | MODERATE |
| HG792016.1 | 109343 | G | A | synonymous_variant | LOW |
| HG792016.1 | 109386 | C | T | missense_variant | MODERATE |
| HG792016.1 | 109387 | G | A | missense_variant | MODERATE |
| HG792016.1 | 109392 | C | T | stop_gained | HIGH |
| HG792016.1 | 109400 | G | A | synonymous_variant | LOW |
| HG792016.1 | 109401 | C | T | missense_variant | MODERATE |
| HG792016,1 | 109402 | C | T | missense_variant | MODERATE |
| HG792016.1 | 109411 | C | T | missense_variant | MODERATE |
| HG792016.1 | 109413 | C | T | stop_gained | HIGH |
| HG792016.1 | 109419 | G | A | missense_variant | MODERATE |
| HG792016.1 | 109424 | C | T | synonymous_variant | LOW |
| HG792016.1 | 109427 | C | T | synonymous_variant | LOW |
| HG792016.1 | 109434 | C | T | missense_variant | MODERATE |
| HG792016.1 | 109435 | G | A | missense_variant | MODERATE |
| HG792016.1 | 109448 | G | A | synonymous_variant | LOW |
| HG792016.1 | 109450 | C | T | missense_variant | MODERATE |
| HG792016.1 | 109459 | C | T | missense_variant | MODERATE |
| HG792016.1 | 109463 | G | A | synonymous_variant | LOW |
| HG792016.1 | 109474 | C | T | missense_variant | MODERATE |
| HG792016.1 | 109485 | C | T | stop_gained | HIGH |
| HG792016.1 | 109497 | C | T | missense_variant | MODERATE |
| HG792016.1 | 109501 | G | A | missense_variant | MODERATE |
| HG792019.1 | 1321186 | C | T | missense_variant | MODERATE |
| HG792019.1 | 1321586 | C | T | synonymous_variant | LOW |
| HG792019.1 | 1321866 | C | G | synonymous_variant | LOW |
| HG792019.1 | 1321897 | C | T | missense_variant | MODERATE |
| HG792019.1 | 1321961 | C | T | missense_variant | MODERATE |
| HG792019.1 | 1322146 | C | T | synonymous_variant | LOW |

| | | | RefCov, | |
|---|---|---|---|---|
| Contig | Gene | GeneID | AltCov | TotalCov |
| HG792015.1 | PROQFM164_S01g002951 | gene2947 | 11.33 | 44 |
| HG792015.1 | PROQFM164_S01g002951 | gene2947 | 16.37 | 53 |
| HG792016.1 | PROQFM164_S02g000051 | gene3710 | 0.0 | 0 |
| HG792016.1 | PROQFM164_S02g000051 | gene3710 | 0.29 | 29 |
| HG792016.1 | PROQFM164_S02g000051 | gene3710 | 0.29 | 29 |
| HG792016.1 | PROQFM164_S02g000051 | gene3710 | 0.29 | 29 |
| HG792016.1 | PROQFM164_S02g000051 | gene3710 | 0.28 | 28 |
| HG792016.1 | PROQFM164_S02g000051 | gene3710 | 0.28 | 28 |
| HG792016.1 | PRGQFM164_S02g000051 | gene3710 | 0.27 | 27 |
| HG792016.1 | PROQFM164_S02g000051 | gene3710 | 0.27 | 27 |
| HG792016.1 | PROQFM164_S02g000051 | gene3710 | 0.27 | 27 |
| HG792016.1 | PROQFM164_S02g000051 | gene3710 | 0.27 | 27 |
| HG792016.1 | PROQFM164_S02g000051 | gene3710 | 0.26 | 26 |
| HG792016.1 | PROQFM164_S02g000051 | gene3710 | 0.26 | 26 |
| HG792016.1 | PROQFM164_S02g000051 | gene3710 | 0.26 | 26 |
| HG792016.1 | PROQFM164_S02g000051 | gene3710 | 0.26 | 26 |
| HG792016.1 | PROQFM164_S02g000061 | gene3710 | 0.20 | 20 |
| HG792016.1 | PROQFM164_S02g000051 | gene3710 | 0.19 | 19 |
| HG792016.1 | PROQFM164_S02g000051 | gene3710 | 0.19 | 19 |
| HG792016.1 | PROQFM164_S02g000051 | gene3710 | 0.18 | 18 |
| HG792016.1 | PROQFM164_S02g000051 | gene3710 | 0.18 | 18 |
| HG792016,1 | PROQFM164_S02g000051 | gene3710 | 0.18 | 18 |
| HG792016.1 | PROQFM164_S02g000051 | gene3710 | 0.18 | 18 |
| HG792016.1 | PROQFM164_S02g000051 | gene3710 | 0.18 | 18 |
| HG792016.1 | PROQFM164_S02g000051 | gene3710 | 0.18 | 18 |
| HG792016.1 | PROQFM164_S02g000051 | gene3710 | 0.19 | 19 |
| HG792016.1 | PROQFM164_S02g000051 | gene3710 | 0.18 | 18 |
| HG792016.1 | PROQFM164_S02g000051 | gene3710 | 0.17 | 17 |
| HG792016.1 | PROQFM164_S02g000051 | gene3710 | 0.17 | 17 |
| HG792016.1 | PRQQFM164_S02g000051 | gene3710 | 0.16 | 16 |
| HG792016.1 | PROQFM164_S02g000051 | gene3710 | 0.16 | 16 |
| HG792016.1 | PROQFM164_S02g000051 | gene3710 | 0.16 | 16 |
| HG792016.1 | PROQFM164_S02g000051 | gene3710 | 0.15 | 15 |
| HG792016.1 | PROQFM164_S02g000051 | gene3710 | 0.14 | 14 |
| HG792016.1 | PROQFM164_S02g000051 | gene3710 | 0.12 | 12 |
| HG792016.1 | PROQFM164_S02g000051 | gene3710 | 0.10 | 10 |
| HG792016.1 | PROQFM164_S02g000051 | gene3710 | 0.10 | 10 |
| HG792019.1 | PROQFM164_S05g000563 | gene10538 | 0.92 | 92 |
| HG792019.1 | PROQFM164_S05g000563 | gene10538 | 0.114 | 114 |
| HG792019.1 | PROQFM164_S05g000563 | gene10538 | 0.65 | 65 |
| HG792019.1 | PROQFM164_S05g000563 | gene10538 | 0.62 | 62 |
| HG792019.1 | PROQFM164_S05g000563 | gene10538 | 0.73 | 73 |
| HG792019.1 | PROQFM164_S05g000563 | gene10538 | 2.33 | 35 |

TABLE B
Differences between reference genome and strain 12A

| | | | | | | | | RefCov, | |
|---|---|---|---|---|---|---|---|---|---|
| Contig | Position | Ref | Alt | Type | Rank | Gene | GeneID | AltCov | TotalCov |
| HG792015.1 | 6997837 | G | A | synonymous_variant | LOW | PROQFM164_S01g002951 | gene2947 | 2.55 | 57 |
| HG792015.1 | 6998275 | T | G | synonymous_variant | LOW | PROQFM164_S01g002951 | gene2947 | 0.103 | 103 |
| HG792015.1 | 6999075 | A | G | missense_variant | MODERATE | PROQFM164_S01g002951 | gene2947 | 1.110 | 111 |
| HG792015.1 | 6999088 | T | C | synonymous_variant | LOW | PROQFM164_S01g002951 | gene2947 | 0.113 | 113 |
| HG792015.1 | 6999238 | A | G | synonymous_variant | LOW | PROQFM164_S01g002951 | gene2947 | 0.81 | 81 |
| HG792015.1 | 6999343 | G | T | synonymous_variant | LOW | PROQFM164_S01g002951 | gene2947 | 0.97 | 97 |
| HG792015.1 | 6999601 | A | G | synonymous_variant | LOW | PROQFM164_S01g002951 | gene2947 | 1.26 | 27 |
| HG792016.1 | 109562 | A | G | synonymous_variant | LOW | PROQFM164_S02g000051 | gene3710 | 0.42 | 42 |
| HG792016.1 | 109794 | A | G | missense_variant | MODERATE | PROQFM164_S02g000051 | gene3710 | 1.71 | 72 |
| HG792019.1 | 1321866 | C | G, T | synonymous_variant | LOW | PROQFM164_S05g000563 | gene10538 | 0,57,4 | 61 |
| HG792019.1 | 1321966 | TG | T | frameshift_variant | HIGH | PROQFM164_S05g000563 | gene10538 | 50.10 | 60 |

TABLE C
Differences between reference genome and strain A22

| Contig | Position | Ref | Alt | Type |
|---|---|---|---|---|
| HG792015.1 | 6999633 | A | ACAACAGCAGCAACAGCGAAGC | conservative_inframe_insertion |
| HG792016.1 | 109127 | C | T | synonymous_variant |
| HG792016.1 | 109130 | C | T | synonymous_variant |
| HG792016.1 | 109138 | C | T | missense_variant |
| HG792016.1 | 109144 | C | T | missense_variant |
| HG792016.1 | 109151 | C | T | splice_region_variant&synonymous_variant |
| HG792016.1 | 109215 | C | T | stop_gained |
| HG792016.1 | 109224 | C | T | stop_gained |
| HG792016.1 | 109242 | C | T | stop_gained |
| HG792016.1 | 109245 | C | T | missense_variant |
| HG792016.1 | 109256 | C | T | synonymous_variant |
| HG792016.1 | 109272 | C | T | missense_variant |
| HG792016.1 | 109554 | C | T | missense_variant |
| HG792016.1 | 109557 | C | T | missense_variant |
| HG792016.1 | 109561 | C | T | missense_variant |
| HG792016.1 | 109575 | C | T | stop_gained |
| HG792016.1 | 109624 | C | T | missense_variant |
| HG792016.1 | 109625 | A | G | synonymous_variant |
| HG792016.1 | 109627 | C | T | missense_variant |
| HG792016.1 | 109640 | C | T | synonymous_variant |
| HG792016.1 | 109643 | C | T | synonymous_variant |
| HG792019.1 | 1321265 | T | C | synonymous_variant |
| HG792019.1 | 1321268 | C | T | synonymous_variant |
| HG792019.1 | 1321292 | T | G | synonymous_variant |
| HG792019.1 | 1321293 | G | T | missense_variant |
| HG792019.1 | 1321297 | C | A | missense_variant |
| HG792019.1 | 1321298 | A | G | synonymous_variant |
| HG792019.1 | 1321760 | C | T | missense_variant |
| HG792019.1 | 1321767 | T | C | synonymous_variant |
| HG792019.1 | 1321850 | T | C | missense_variant |
| HG792019.1 | 1321857 | A | G | synonymous_variant |
| HG792019.1 | 1321863 | G | A | synonymous_variant |
| HG792019.1 | 1321866 | C | T | synonymous_variant |
| HG792019.1 | 1321872 | A | G | synonymous_variant |
| HG792019.1 | 1321876 | G | A | synonymous_variant |
| HG792019.1 | 1321884 | C | T | synonymous_variant |
| HG792019.1 | 1321893 | G | T | synonymous_variant |
| HG792019.1 | 1321896 | A | G | synonymous_variant |
| HG792019.1 | 1321920 | C | A | synonymous_variant |
| HG792019.1 | 1321966 | T | C | missense_variant |
| HG792019.1 | 1322028 | G | A | synonymous_variant |

| | | | | RefCov, | |
|---|---|---|---|---|---|
| Contig | Rank | Gene | GeneID | AltCov | TotalCov |
| HG792015.1 | MODERATE | PROQFM164_S01g002951 | gene2947 | 2.12 | 14 |
| HG792016.1 | LOW | PROQFM164_S02g000051 | gene3710 | 78.59 | 137 |
| HG792016.1 | LOW | PROQFM164_S02g000051 | gene3710 | 76.60 | 136 |
| HG792016.1 | MODERATE | PROQFM164_S02g000051 | gene3710 | 77.59 | 136 |
| HG792016.1 | MODERATE | PROQFM164_S02g000051 | gene3710 | 73.60 | 133 |
| HG792016.1 | LOW | PROQFM164_S02g000051 | gene3710 | 70.60 | 130 |
| HG792016.1 | HIGH | PROQFM164_S02g000051 | gene3710 | 76.69 | 145 |
| HG792016.1 | HIGH | PROQFM164_S02g000051 | gene3710 | 75.70 | 145 |
| HG792016.1 | HIGH | PROQFM164_S02g000051 | gene3710 | 80.73 | 153 |
| HG792016.1 | MODERATE | PROQFM164_S02g000051 | gene3710 | 78.73 | 152 |
| HG792016.1 | LOW | PROQFM164_S02g000051 | gene3710 | 79.75 | 154 |
| HG792016.1 | MODERATE | PROQFM164_S02g000051 | gene3710 | 75.81 | 156 |
| HG792016.1 | MODERATE | PROQFM164_S02g000051 | gene3710 | 62.92 | 154 |
| HG792016.1 | MODERATE | PROQFM164_S02g000051 | gene3710 | 61.92 | 153 |
| HG792016.1 | MODERATE | PROQFM164_S02g000051 | gene3710 | 62.92 | 154 |
| HG792016.1 | HIGH | PROQFM164_S02g000051 | gene3710 | 64.90 | 154 |
| HG792016.1 | MODERATE | PROQFM164_S02g000051 | gene3710 | 64.88 | 152 |
| HG792016.1 | LOW | PROQFM164_S02g000051 | gene3710 | 65.86 | 151 |
| HG792016.1 | MODERATE | PROQFM164_S02g000051 | gene3710 | 66.86 | 152 |
| HG792016.1 | LOW | PROQFM164_S02g000051 | gene3710 | 60.87 | 147 |
| HG792016.1 | LOW | PROQFM164_S02g000051 | gene3710 | 60.87 | 147 |
| HG792019.1 | LOW | PROQFM164_S05g000563 | gene10638 | 85.20 | 105 |
| HG792019.1 | LOW | PROQFM164_S05g000563 | gene10538 | 85.20 | 105 |
| HG792019.1 | LOW | PROQFM164_S05g000563 | gene10538 | 88.8 | 96 |
| HG792019.1 | MODERATE | PROQFM164_S05g000563 | gene10538 | 88.8 | 96 |
| HG792019.1 | MODERATE | PROQFM164_S05g000563 | gene10538 | 89.8 | 97 |
| HG792019.1 | LOW | PROQFM164_S05g000563 | gene10538 | 88.8 | 96 |
| HG792019.1 | MODERATE | PROQFM164_S05g000563 | gene10538 | 71.81 | 152 |
| HG792019.1 | LOW | PROQFM164_S05g000563 | gene10538 | 68.82 | 150 |
| HG792019.1 | MODERATE | PROQFM164_S05g000563 | gene10538 | 52.11 | 63 |
| HG792019.1 | LOW | PROQFM164_S05g000563 | gene10538 | 52.11 | 63 |
| HG792019.1 | LOW | PROQFM164_S05g000563 | gene10538 | 52.12 | 64 |
| HG792019.1 | LOW | PROQFM164_S05g000563 | gene10538 | 53.13 | 66 |
| HG792019.1 | LOW | PROQFM164_S05g000563 | gene10538 | 52.14 | 66 |
| HG792019.1 | LOW | PROQFM164_S05g000563 | gene10538 | 52.16 | 68 |
| HG792019.1 | LOW | PROQFM164_S05g000563 | gene10538 | 56.20 | 76 |
| HG792019.1 | LOW | PROQFM164_S05g000563 | gene10538 | 56.21 | 77 |
| HG792019.1 | LOW | PROQFM164_S05g000563 | gene10538 | 56.24 | 80 |
| HG792019.1 | LOW | PROQFM164_S05g000563 | gene10538 | 64.32 | 96 |
| HG792019.1 | MODERATE | PROQFM164_S05g000563 | gene10538 | 57.41 | 98 |
| HG792019.1 | LOW | PROQFM164_S05g000563 | gene10538 | 70.48 | 118 |

TABLE D
Differences between reference genome and strain B20

| | | | | | | RefCov, | |
|---|---|---|---|---|---|---|---|
| Ref | Alt | Type | Rank | Gene | GeneID | AltCov | TotalCov |
| ACAGCGAAGC | A | disruptive_inframe_deletion | MODERATE | PROQFM164_S01g002951 | gene2947 | 6.17 | 23 |
| T | A | synonymous_variant | LOW | PROQFM164_S02g000051 | gene3710 | 0.44 | 44 |
| A | G | missense_variant | MODERATE | PROQFM164_S02g000051 | gene3710 | 0.46 | 46 |
| T | C | synonymous_variant | LOW | PROQFM164_S02g000051 | gene3710 | 0.46 | 46 |
| A | G | synonymous_variant | LOW | PROQFM164_S02g000051 | gene3710 | 0.43 | 43 |
| T | G | missense_variant | MODERATE | PROQFM164_S02g000051 | gene3710 | 0.43 | 43 |
| A | G | missense_variant | MODERATE | PROQFM164_S02g000051 | gene3710 | 0.39 | 39 |
| T | C | synonymous_variant | LOW | PROQFM164_S02g000051 | gene3710 | 0.21 | 21 |
| G | A | missense_variant | MODERATE | PROQFM164_S02g000051 | gene3710 | 0.19 | 19 |
| C | G | missense_variant | MODERATE | PROQFM164_S02g000051 | gene3710 | 0.17 | 17 |
| A | C | synonymous_variant | LOW | PROQFM164_S02g000051 | gene3710 | 0.17 | 17 |
| C | T | missense_variant | MODERATE | PROQFM164_S02g000051 | gene3710 | 0.16 | 16 |
| G | A | missense_variant | MODERATE | PROQFM164_S02g000051 | gene3710 | 0.13 | 13 |
| CTTCT | C | frameshift_variant | HIGH | PROQFM164_S02g000051 | gene3710 | 0.8 | 8 |
| T | C | synonymous_variant | LOW | PROQFM164_S02g000051 | gene3710 | 0.14 | 14 |
| A | G | synonymous_variant | LOW | PROQFM164_S02g000051 | gene3710 | 0.77 | 77 |
| G | A | synonymous_variant | LOW | PROQFM164_S02g000051 | gene3710 | 0.77 | 77 |
| C | G | synonymous_variant | LOW | PROQFM164_S02g000051 | gene3710 | 0.76 | 76 |
| C | T | synonymous_variant | LOW | PROQFM164_S02g000051 | gene3710 | 0.72 | 72 |
| C | T | synonymous_variant | LOW | PROQFM164_S02g000051 | gene3710 | 0.65 | 65 |
| T | C | synonymous_variant | LOW | PROQFM164_S02g000051 | gene3710 | 0.62 | 62 |
| A | T | missense_variant | MODERATE | PROQFM164_S02g000051 | gene3710 | 0.59 | 59 |
| A | G | synonymous_variant | LOW | PROQFM164_S02g000051 | gene3710 | 0.58 | 58 |
| G | A | missense_variant | MODERATE | PROQFM164_S02g000051 | gene3710 | 0.58 | 58 |
| A | C | synonymous_variant | LOW | PROQFM164_S02g000051 | gene3710 | 0.12 | 12 |
| C | G | synonymous_variant | LOW | PROQFM164_S02g000051 | gene3710 | 0.14 | 14 |
| C | A | missense_variant | MODERATE | PROQFM164_S02g000051 | gene3710 | 0.26 | 26 |
| T | G | missense_variant | MODERATE | PROQFM164_S02g000051 | gene3710 | 0.27 | 27 |
| G | C | missense_variant | MODERATE | PROQFM164_S02g000051 | gene3710 | 0.28 | 28 |
| A | G | missense_variant | MODERATE | PROQFM164_S02g000051 | gene3710 | 0.31 | 31 |
| G | A | missense_variant | MODERATE | pROQFM164_S02g000051 | gene3710 | 0.38 | 38 |
| G | A | synonymous_variant | LOW | PROQFM164_S02g000051 | gene3710 | 0.46 | 46 |
| T | C | synonymous_variant | LOW | PROQFM164_S05g000563 | gene10538 | 84.8 | 92 |
| A | G | missense_variant | MODERATE | PROQFM164_S05g000563 | gene10538 | 80.17 | 97 |
| A | G | synonymous_variant | LOW | PROQFM164_S05g000563 | gene10538 | 83.19 | 102 |
| G | T | missense_variant | MODERATE | PROQFM164_S05g000563 | gene10538 | 116.13 | 129 |
| G | A | synonymous_variant | LOW | PROQFM164_S05g000563 | gene10538 | 118.13 | 131 |
| A | G | synonymous_variant | LOW | PROQFM164_S05g000563 | gene10538 | 115.11 | 126 |
| A | G | synonymous_variant | LOW | PROQFM164_S05g000563 | gene10538 | 158.36 | 194 |
| T | C | missense_variant | MODERATE | PROQFM164_S05g000563 | gene10538 | 159.35 | 194 |
| C | T | synonymous_variant | LOW | PROQFM164_S05g000563 | gene10538 | 159.35 | 194 |
| C | A, G | synonymous_variant | LOW | PROQFM164_S05g000563 | gene10538 | 68,90,34 | 192 |
| TG | CG, T | frameshift_variant | HIGH | PROQFM164_S05g000563 | gene10538 | 73,73,26 | 172 |
| G | A | synonymous_variant | LOW | PROQFM164_S05g000563 | gene10538 | 144.21 | 165 |
| G | A, T | synonymous_variant | LOW | PROQFM164_S05g000563 | gene10538 | 74,64,19 | 157 |
| T | C | synonymous_variant | LOW | PROQFM164_S05g000563 | gene10538 | 73.53 | 126 |
| C | T | synonymous_variant | LOW | PROQFM164_S05g000563 | gene10538 | 70.48 | 118 |
| G | A | missense_variant | MODERATE | PROQFM164_S05g000563 | gene10538 | 70.46 | 116 |
| A | C | synonymous_variant | LOW | PROQFM164_S05g000563 | gene10538 | 75.43 | 118 |
| C | T | missense_variant | MODERATE | PROQFM164_S05g000563 | gene10538 | 71.32 | 103 |
| C | T | missense_variant | MODERATE | PROQFM164_S05g000563 | gene10538 | 70.32 | 102 |
| T | A | missense_variant | MODERATE | PROQFM164_S05g000563 | gene10538 | 70.31 | 101 |
| C | G | missense_variant | MODERATE | PROQFM164_S05g000563 | gene10538 | 70.27 | 97 |
| G | A | synonymous_variant | LOW | PROQFM164_S05g000563 | gene10538 | 73.27 | 100 |
| T | C | missense_variant | MODERATE | PROQFM164_S05g000563 | gene10538 | 73.27 | 100 |
| C | T | missense_variant | MODERATE | PROQFM164_S05g000563 | gene10538 | 75.20 | 95 |
| T | C | synonymous_variant | LOW | PROQFM164_S05g000563 | gene10538 | 74.20 | 94 |
| A | G | synonymous_variant | LOW | PROQFM164_S05g000563 | gene10538 | 77.19 | 96 |

A second aspect of the present invention provides derivative Penicillium strains having altered spore colour compared to a corresponding unmodified (control) strain.

Almost all Penicillium species naturally produce spores exhibiting a green to blue to grey colour on standard growth media (for example, CYA, MEA, PDA, ACM), although the precise colour can vary somewhat depending on the growth media, with a few species, such as P. camemberti, producing white spores.

The altered spore colour may result from a change in the pigment development pathway of the Penicillium strain. However, some spores have been known to change colour without any change in currently known pigment developmental pathways.

The main pigment development pathway of Penicillium and related Aspergillus species is well known and is described in, for example, Woo et al., 2010 (High diversity of polyketide synthase genes and the melanin biosynthesis gene cluster in Penicillium marneffei. FEBS Journal 277; 3750-3758) and Krijgsheld et al., 2013 (Development in Aspergillus. Studies in Mycology 74: 1-29) and is also shown in FIG. 9 herein.

The term “altered pigment development pathway” as defined herein is a change in the pigment (melanin) development pathway compared to the pigment development pathway of an unmodified strain and which change causes the derivative to produce spores of a different colour compared to the corresponding unmodified strain.

Reference herein to a “derivative” strain is taken to mean a Penicillium strain which has been modified to produce spores which differ in colour compared to the strain from which it is derived. Reference herein to being “derived from” any given strain encompasses being physically derived from that strain as well as methods for artificial sequence synthesis.

The terms “spores” and “conidia” are used interchangeably herein.

In one embodiment, the derivative strain produces spores which are not the typical green or blue-green colour associated with blue cheese.

According to one embodiment, the derivative Penicillium strains produce spores which may be any one or more of the following colours: light olive-brown, brown, reddish-brown-pink, intense to light blue, green and albino. The light olive-brown, brown, reddish-brown-pink, intense to light blue, green or albino spore colour may result from an altered pigment development pathway compared to a corresponding unmodified (control) strain.

There are many ways of determining the colour of fungal spores, but typically this is done by eye. Colour may also be scored using a suitable chart, such as the Methuen colour plate (Methuen Handbook of Colour (second edition), by A. Kornerup & J H Wanscher, published by Methuen Publishing (1967)). For example, the light olive-brown strains may be encompassed by Methuen colour plate ranges 2-5; brown by Methuen colour plate ranges 6-7; reddish-brown-pink by Methuen colour plate ranges 8-10; intense to light blue by Methuen colour plate ranges 20-24; green by Methuen colour plate ranges 26-30; and albino being characterised by an absence of colour.

According to one embodiment, the derivative Penicillium strains produce spores which are not blue-green in colour (for example as encompassed by “Methuen” colour plate 25). According to one embodiment, the derivative strains produce spores which are not albino.

According to an embodiment, the Penicillium strain to be modified is a Penicillium strain as defined in any one or more of (a) to (g) in the first aspect of the invention. For example, the derivative may be a derivative of strain 5A, 12A, A22 or B20, which derivative produces spores of a different colour to the spore colour of 5A, 12A, A22 or B20. This change in colour may be due to a change in the pigment development pathway and/or may be due to the growth media used.

The derivative may be a variant of strain 5A, 12A, A22 or B20 having at least 99.0%, 99.1%, 99.2%, 99.3%, 99.4%, 99.5%, 99.6%, 99.7%, 99.8%, 99.9% or more sequence identity thereto on a DNA level and having the capacity to produce spores which differ in colour compared to the corresponding unmodified strain.

The derivative may have the same or similar biological and functional characteristics as the strain from which it was derived. The derivative strain may have a different or similar organoleptic, for example flavour profile, to the strain from which it was derived. The derivative may have similar or different activities or levels of various enzymes and/or levels of certain secondary metabolites to the strain from which it was derived.

Derivative strains having an altered pigment development pathway may show altered (for example, increased or decreased or substantially eliminated) expression of one or more of the following metabolites involved in melanin production: heptaketide naphthopyrone; 1,3,6,8-tetrahydroxynaphthalene; scytalone; 1,3,8-trihydroxynaphthalene; vermelone; 1,8-dihydroxynaphthalene or other metabolites produced as a result of the action of one or more of the following genes: abr1 (SEQ ID NO: 1), abr2 (SEQ ID NO: 2), alb1 (SEQ ID NO: 3), arp1 (SEQ ID NO: 4), arp2 (SEQ ID NO: 5), ayg1 (SEQ ID NO: 6). The change typically leads to the development of spores having a pigment which differs from the spore colour of the corresponding unmodified strain (control). The pigment may be any colour which differs from that of the control. The colour may optionally be light olive-brown, brown, reddish-brown-pink, intense to light blue, green and albino.

The pigment development pathway of Penicillium strains may be altered using mutagenesis, through for example U.V., X-ray or chemical mutagenesis. Other methods to alter the pigment development pathway include, without limitation, various methods for editing nucleic acid, for example to cause gene knockout, knock-in or expression of a gene to be downregulated or overexpressed, or to introduce mutations in the form of one or more deletions, insertions or substitutions. For example, use of various nuclease systems, such as zinc finger nucleases (ZFN), transcription activator-like effector nucleases (TALEN), meganucleases, or combinations thereof are known in the art for editing nucleic acid and may be used in the present invention. In recent times, the clustered regularly interspersed short palindromic repeats (CRISPR)/CRISPR-associated (Cas) (CRISPR/Cas) nuclease system has become more commonly used for genome engineering. The CRISPR/Cas system is detailed in, for example WO2013/176772, WO2014/093635 and WO2014/089290.

For example, a CRISPR/Cas9 may include a guide RNA (gRNA) sequence with a binding site for Cas9 and a targeting sequence specific for the area to be modified. The Cas9 binds the gRNA to form a ribonucleoprotein that binds and cleaves the target area. In addition to the CRISPR/Cas 9 platform (which is a type II CRISPR/Cas system), alternative systems exist including type I CRISPR/Cas systems, type III CRISPR/Cas systems, and type V CRISPR/Cas systems. Any of the above CRISPR systems may be used to alter the pigment development pathway of Penicillium strains by, for example, altering one or more of the following genes: Alb1, Ayg1, Arp2, Arp1, Abr1, Abr2. The aforementioned genes may be represented by SEQ ID NO: 1 (abr1), SEQ ID NO: 2 (abr2), SEQ ID NO: 3 (alb1), SEQ ID NO: 4 (arp1), SEQ ID NO: 5 (arp2), SEQ ID NO: 6 (ayg1) or by the corresponding gene in the strain to be modified or by a sequence having at least 99.0%, 99.1%, 99.2%, 99.3%, 99.4%, 99.5%, 99.6%, 99.7%, 99.8%, 99.9% or more sequence identity on a DNA or protein level to any of SEQ ID Nos 1 to 6 or to a protein encoded by any one of SEQ ID Nos 1 to 6.

Therefore, one of the aforementioned techniques (or other suitable technique) may be used in a method for the production of Penicillium strains having conidia (spores) of a colour or colours that differs from that of the control (the strain selected for modification). The colour of the resulting derivative strain may be light olive-brown, brown, reddish-brown-pink, pink, green, intense blue to light blue and albino, typically as a result of mutation in genes in the pigment biosynthetic pathway.

A preferred method comprises mutating Penicillium strains by exposing fungal material, such as spores or hyphae, to UV irradiation, or to X-rays or to a chemical mutagen or to any other suitable mutagen. Normally a dose response curve will be determined and then conditions identified where fungal material shows a survival rate up to 20% of original material. Such strains surviving the mutagenesis would be expected to have single or multiple mutations in the genome, which might range from single base pair changes in the DNA sequence through to possible gross chromosomal rearrangements.

The method of producing a derivative Penicillium strain may comprise the step of mutating the strain to change the colour of the spores produced. The strain to be mutated (or otherwise modified using other techniques) may be any strain and may have been produced through asexual or sexual reproduction. The method may be carried out as a standalone method or may be carried out after a sexual cross. The method may be used to produce strains with changed spore colour compared to the unmodified strain. For example, the derivative strains may produce spores of light olive-brown, brown, reddish-brown, reddish-brown-pink, pink, green, albino, intense blue to light blue in colour. Optionally, the method does not produce albino spores. Optionally the method does not produce spores having the conventional green or blue-green colour of known blue cheeses. Such strains may then be used to produce cheeses with different coloured veins and/or surfaces, either of single colours or multiple colours.

The altered spore colour of the derivative may additionally or alternatively be achieved by growing the strains to be modified on suitable growth media (for example potato dextrose agar or milk amended agar media).

According to a third aspect of the present invention, there is provided a method of altering the colour of spores of a Penicillium strain, comprising altering the pigment development pathway compared to a control (unmodified strain). The altering of the pigment development pathway may be carried out by any one or more of: U.V., X-ray or chemical mutagenesis, gene editing techniques, gene transformation (for example, to overexpress or downregulate one or more of the following genes: Alb1, Ayg1, Arp2, Arp1, Abr1, Abr2). The aforementioned genes may be represented by SEQ ID NO: 1 (abr1), SEQ ID NO: 2 (abr2), SEQ ID NO: 3 (alb1), SEQ ID NO: 4 (arp1), SEQ ID NO: 5 (arp2), SEQ ID NO: 6 (ayg1) or by the corresponding gene in the strain to be modified or by a sequence having at least 99.0%, 99.1%, 99.2%, 99.3%, 99.4%, 99.5%, 99.6%, 99.7%, 99.8%, 99.9% or more sequence identity on a DNA level to any of SEQ ID NOs 1 to 6 or to a protein encoded by any one of SEQ ID Nos 1 to 6.

The method also comprises the step of selecting for progeny having spores of altered colour compared to the unmodified strain.

The invention therefore provides, a method of altering the colour of spores of a Penicillium strain, comprising altering the pigment development pathway compared to a corresponding unmodified strain and optionally selecting for progeny having spores of altered colour compared to the unmodified strain. The Penicillium strain is preferably a strain of Penicillium roqueforti. According to one embodiment, the Penicillium strain to be modified is strain 5A, 12A, A22 or B20 or a strain having at least 99.0%, 99.1%, 99.2%, 99.3%, 99.4%, 99.5%, 99.6%, 99.7%, 99.8%, 99.9% or more sequence identity to the genomic DNA sequence of strains 5A, 12A, A22 and B20. The genomic sequence for the aforementioned strains is provided as SEQ ID NOs: 7-54 (5A), SEQ ID NOs: 55-102 (12A), SEQ ID NOs 103-150 (A22) and SEQ ID NOs 151-198 (B20). The at least 99.0% sequence identity may be with reference to the SEQ ID NO provided herein or to the genomic sequence of the strain as deposited under the Budapest Treaty at the Westerdijk Fungal Biodiversity Institute: CBS 145358 (5A), CBS 145359 (12A), CBS 145360 (A22) and CBS 145361 (B20).

A fourth aspect of the present invention provides fungal spores obtainable (or obtained) by a method according to the third aspect of the invention, which spores differ in colour compared to spores of the corresponding unmodified (or control) strain. Also provided are fungal strains comprising such spores.

The novel strains listed under points (a) to (g) in the first aspect of the invention and variants and derivatives thereof may have a different organoleptic, for example flavour profile, compared to existing production strains or compared to the organoleptic profile of either one or both parents or an unmodified strain.

The flavour of mould-ripened cheeses is determined by a number of volatile and non-volatile chemicals as listed in Table 1 below. Of these, methyl ketones such as 2-undecanone, 2-nonanone and 2-heptanone are particularly responsible for blue-cheese flavour, but other ketones such as 2-hexanone, 2-pentanone and 2-butanone together with alcohols such as 1-octen-3-ol and 2-heptanol also contribute to flavour. For example, Roquefort cheese characteristically contains 2-heptanone as the most abundant ketone followed by 2-nonanone. Whereas in Stilton cheese the dominant ketones are 2-heptanone, 2-butanone and 2-pentanone.

TABLE 1

| Volatile | Non-volatile |
|---|---|
| Methyl Ketones | Organic acids |
| Fatty acids (short chain) | Fatty acids (long chain) |
| 2-Alkanols | Fatty acid soaps |
| Aldehydes | Alcohols |
| Amines | Amino acids |
| Esters | Secondary amines |
| Sulfides | Peptides |
| Lactones | |

The levels of production of volatile chemicals can be determined using methods such as gas chromatography mass spectroscopy (GC-MS). Data can then be analysed by principal component analysis to indicate the flavour profiles given by strains of Penicillium roqueforti, as determined by the relative production of 15 or more volatile chemicals which are assessed (for example, 2-undecanone, 2-decanone, 2-pentanol, 2-nonanol, 2-nonanone, 2-heptanol, 8-nonen-2-one, 2-octanone, 2-pentanone, 2-heptanone, 2-hexanione, 1-pentanol, 2-methy butanal, 3-methyl butanal, isoamyl isobutanol, 3-methyl butanol, 3-octanone). By determining the relative levels of volatile chemicals produced, the identity of strains used in production of Roquefort, Stilton and Gorganzola cheeses can be determined. This technique can also allow the identification of novel strains of Penicillium roqueforti produced by sexual crossing, which occupy novel flavour spaces due to differential levels of production of the volatile flavour chemicals and differing enzyme levels and activity. For example, novel strains can be generated producing high or low levels of 2-undecanone and/or 2-nonanone with relatively low production of 3-octanone and/or 3-methyl butanol, relative to either one or both parental strains or unmodified or known strain. Similarly other novel strains contain high levels of 2-pentanol and/or 2-heptanol with either high or low levels of 2-undecanone and 2-nonanol, relative to either one or both parental strains or unmodified or known strain. Strains may show a 2 to 20-fold or more difference in levels of 2-undecanone production compared to either one or both parent strains or unmodified or known strain. Strains may show a 2 to 20-fold, or more difference in levels of 2-nonanone production compared to either one or both parent strain or unmodified or known strain. Strains may show a 2 to 20-fold or more difference in levels of 3-octanone production compared to either parent strain or unmodified or known strain. Strains may show a 2 to 20-fold or more difference in levels of 3-methyl butanol production compared to either one or both parent strain or unmodified or known strain. Strains may show a 2 to 20-fold or more difference in levels of 2-heptanol production compared to either one or both parent strains or unmodified or known strains.

The altered levels and/or activity of enzymes such as proteases and/or lipases may also have an effect on a final product. For example, if used in a cheese, a fungal strain with increased protease activity may result in faster maturation of the cheese, thereby reducing production costs. Similarly, strains with increased lipase activity might also result in faster maturation, and at the same time exhibit a novel flavour spectrum and other organoleptic qualities due to increased production of volatile flavour compounds.

The novel strains listed under points (a) to (g) in the first aspect of the invention and variants and derivatives thereof suitably have a novel organoleptic, for example flavour, profile compared to one or both parents in the case of sexually derived strains, or have different flavour profiles compared to existing cheese production strains in the case of strains isolated from the environment.

Strains 5A, 12A, A22 and B20 produce blue-green coloured spores. Strain A22 when used in cheese production produces an intense-flavoured blue cheese, strain B20 a mild-flavoured blue cheese, strain 5A a classic yet tangy/fruity blue-cheese flavour, whilst strain 12A produces an aromatic, earthy flavour blue cheese. Although the final flavour of the cheese will also depend on other factors such as the type of milk, method of cheese production etc., (aside from the strain of Penicillium roqueforti used), the flavour contributed by the Penicillium strain (for example, intense, mild, tangy, fruity, earthy etc.) will remain unchanged.

The novel strains listed under points (a) to (g) in the first aspect of the invention and variants and derivatives thereof are strains of Penicillium. Any of the commercially-relevant Penicillium strains, particularly those suitable for use in food products, particularly in the production of cheese may be useful in the invention. For example, the Penicillium may be Penicillium roqueforti, Penicillium paneum, Penicillium glaucum or Penicillium camemberti. The strain may be a Stilton-type, Roquefort-type or Gorgonzola-type strain.

According to a fifth aspect of the present invention, there is provided use in industry of the novel strains listed under points (a) to (g) in the first aspect of the invention and variants and derivatives thereof. The novel strains, variants and derivatives thereof are not only useful in the food industry, for example in cheese production, but may also find use in nutraceutical production; agricultural applications; bioremediation; enzyme production (particularly in the production of lipases and proteases); fermentation process, for example in the production of biofuels, or in hay or silage production; or in the production of or in animal feed.

For example, the novel strains, variants and derivatives thereof may find use in bioremediation, for example in the degradation of various herbicides, fungicides etc. Other bioremediation applications include treating metal accumulation and possible metal recovery of contaminated soil.

The Penicillium strains of the invention may find use in fermentation processes, for example in the degradation of biomasses, for the recovery of cellulose, sugars and/or for ethanol production. Penicillium roqueforti, for example, has been described in Mioso et al., 2014, (Journal of Applied Microbiology 118, 781-791) as a natural candidate for industrial biotechnological production given its many other favourable fermentation characteristics, such as its tolerance for growing at low pH values and the ability to use both pentoses and hexoses as substrates.

The novel strains, variants and derivatives thereof may be used in the production of enzymes, not just in the production of lipases and proteases, but also in the production of decarboxylases and deaminases. The enzymes produced by Penicillium roqueforti may be used in diverse industries, including biotechnology, in food, chemical, pharmaceutical and agricultural industries.

The novel strains, variants and derivatives thereof may find use in applications where increased digestibility of a substrate is required, such as in agricultural applications, in feed stocks and silaging.

The novel strains, variants and derivatives may find use in in the food industry, not just in cheese production, but more widely for food preparation where the fermentative and enzymatic properties of the strains may be used.

Strains produced by the methods of the invention may also be used in the production of other useful metabolites. For example, it has been suggested that Penicillium roqueforti might produce health-promoting metabolites that contribute to the so-called ‘French paradox’. Therefore, the sexual crossing process could be used to produce sexual offspring which could be selected for the production of increased levels of such beneficial metabolites.

According to a sixth aspect of the invention, there is provided a composition comprising any of the aforementioned novel strains, variants and derivatives thereof and an acceptable carrier. In certain embodiments, the carrier is an agriculturally acceptable carrier. In some embodiments, the carrier is suitable for nutraceutical administration. In further embodiments, the carrier is suitable for use for food applications. In further embodiments, the carrier is suitable for bioremediation applications.

In certain embodiments, the aforementioned composition comprises at least one additional bacteria or fungus, selected according to the end application.

According to a seventh aspect of the present invention, there is provided a food product produced by or comprising the strain and/or variant and/or derivative of the invention. The food product is preferably a dairy product. The term ‘dairy product’ is not limited to milk from cows, but also encompasses sheep, goat, buffalo and other sources of milk. The dairy product is preferably cheese, optionally a mould-ripened cheese. The food product may be an item to be used in the manufacture of a food product, such as a starter culture or (dry) cheese ingredient, or may be in its final “ready-to-consume” form. The food product may be a starter culture, a cheese, cheese sauce, cheese spread, dry cheese ingredient etc.

The cheese may be a hard or soft cheese. The fungal strains may be contained in veins in the cheese and/or may be found on the surface of the cheese. The Penicillium may be in the veins of a cheese, in particular if the strain has been mutated or otherwise modified to produce spores of a different colour than that of the wild type strain or other control. Commonly this type of cheese is referred to as a blue cheese due to the colour of the fungal spores in the veins. The cheese may be ripened by a Penicillium internal and/or external mould.

The invention also provides a cheese or cheese product with veins of Penicillium wherein the Penicillium strain produces spores which are light olive-brown, brown, light brown, olive brown, reddish, reddish-brown, reddish-brown-pink, pink, green, intense blue to light blue or albino. The cheese (or cheese product) may comprise multiple strains to produce a variety of colours in the end product. The cheese or cheese product may comprise the strain and/or variant and/or derivative in addition to other moulds.

The cheese may be selected from a Roquefort, Bleu de Bresse, Bleu du Vercors-Sassenage, Brebiblu, Cabrales, Cambozola (Blue Brie), Cashel Blue, Danish blue, blue Cheddar, Fourme d′Ambert, Fourme de Montbrison, Lanark Blue, Maytag Blue, Strathdon Blue, Blue Murder, Shropshire Blue, Dorset blue vinney, Brighton blue, Moyden's Wrekin blue, picos blue, cabrales, rokpol, dolcelatte, Stilton, and some varieties of Bleu d′Auvergne, a Gorgonzola, a Brie, a Camembert, as well as other blue cheeses or any other cheese ripened by a Penicillium internal or external mould.

The cheese or cheese product may be any known cheese or cheese product which has been produced using a novel strain, variant or derivative according to the invention, or into which such strain(s), variant(s) or derivative(s) have been introduced. Use of the strain may impart a colour or multiple colours to the resultant product.

The cheese or cheese product may have any one or more of the following sensorial and/or organoleptic qualities attributed by novel strain, variant or derivative according to the invention: improved mouthfeel, rounded taste, bland taste, mild taste, strong or intense taste, piquant taste, tangy taste, fruity taste, aromatic flavour, earthy flavour, strong aroma, medium aroma, mild aroma.

According to an eight aspect of the present invention, there is provided a method of producing strains of Penicillium by sexual reproduction, comprising the steps of:
1. crossing complementary MAT1-1 and MAT1-2 strains of Penicillium;
2. inoculating the isolates onto an agar medium;
3. incubating the inoculated media plates at a temperature of between 10-20° C.; and
4. obtaining ascospores.

Step one of the method involves obtaining isolates of complementary MAT1-1 and MAT1-2 mating types and crossing the same (one isolate expresses MAT1-1 and the other expresses MAT1-2 genes). The mating type of Penicillium isolates can be readily determined by using a polymerase chain reaction (PCR)-based DNA diagnostic test.

Penicillium strains useful in the method of the invention may be any suitable complementary mating type. For example, the strains of Penicillium used may be selected from Penicillium roqueforti, Penicillium paneum, Penicillium glaucum and Penicillium camemberti. In a particular embodiment, the strains of Penicillium used may be selected from Penicillium roqueforti and/or Penicillium paneum.

The second step of the method involves inoculating the isolates. Any suitable agar medium may be used, for example, an oatmeal agar medium. Preferably, the isolates are inoculated between about 1 to about 3 cm apart, suitably in such a way as to result in the fungal hyphae growing towards each other to form a barrage (as discussed in O'Gorman et al., (2009) Nature 457: 471-474).

Step three of the method involves incubating, for example, at between about 10 to about 20° C. in the dark for a period between about 3 to about 24 weeks, optionally between about 3 to about 12 weeks or between about 3 to about 6 weeks. The incubation temperature of between 10-20° C. advantageously increased the chance of mating compared to conventional methods. Preferably, the incubation takes place at a temperature of between 10-20° C., optionally between 10-15° C. or between 15-20° C. Over the course of this time, the formation of sexual structures known as cleistothecia can be observed. These are normally in the barrage zones, although they can be more widespread on the growth plates.

The final step of the method involves obtaining ascospores from the cleistothecia, for example through dissection. Ascospores represent the novel sexual offspring strains, which can in turn be used to establish new fungal colonies.

A ninth aspect of the invention relates to ascospores obtainable (or obtained) by a method according to the eight aspect of the invention. The invention also relates to a strain of Penicillium obtainable (or obtained) by any method of the invention.

According to a tenth aspect of the present invention, there is provided a method of screening for strains having flavour profiles comparable to strains B20, A22, 5A, 12A or variants or derivatives thereof:
a. carrying out GC-MS analysis of strains B20, A22, 5A or 12A, variants or derivatives thereof as defined herein;
b. carrying out GC-MS analysis of one or more query strains;
c. selecting those strains in (b) which group together with strains B20, A22, 5A or 12A, variants or derivatives thereof as shown in the GC-MS readings, as may be further analysed by principal component analysis or the like.

The skilled man will appreciate that all preferred features and aspects of any aspect of the invention can be applied to all aspects of the invention.

Throughout the description and claims of this specification, the words “comprise” and “contain” and variations of the words, for example “comprising” and “comprises”, mean “including but not limited to”, and do not exclude other components, integers or steps. Moreover the singular encompasses the plural unless the context otherwise requires: in particular, where the indefinite article is used, the specification is to be understood as contemplating plurality as well as singularity, unless the context requires otherwise.

Preferred features of each aspect of the invention may be as described in connection with any of the other aspects. Within the scope of this application it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible.

### BRIEF DESCRIPTION OF THE DRAWINGS

One or more embodiments of the invention will now be described, by way of example only, with reference to the accompanying drawings, in which:

FIG. 1 illustrates the positioning of aliquots of fungal spore suspensions on 9 cm Petri dishes of oatmeal agar (1=isolate of MAT1-1 genotype; 2=isolate of MAT1-2 genotype).

FIG. 2 illustrates the presence of sexual reproductive structures in P. roqueforti. (a-d) Paired cultures of 74-88×74-92 on oatmeal agar with yellow cleistothecia (arrowed) produced along the barrage zones following four weeks incubation at 10° C. Scale bar, 300 μm.

FIG. 3 provides evidence for meiotic recombination in P. roqueforti. Segregation patterns of a RAPD-PCR amplicon parental isolates (P1, P2) and 12 ascospore progeny (1-12) from the cross 74-92 (P1)×74-88 (P2), using primer OPW10 MM, molecular weight marker; C, water control; kb, kilobase. Arrow indicates the diagnostic RAPD band. Lane headings: MAT1-1 (red) and MAT1-2 (green) genotypes.

FIG. 4 shows a principle component analysis to illustrate that the sexual offspring from a P. roqueforti cross (labelled 1-30) show production of combinations of flavour volatiles which differ from those of the parental strains (Parent 5 and Parent 18) derived from Gorgonzola and Roquefort cheeses.

FIG. 5 shows a principle component analysis to illustrate that the sexual offspring from a P. roqueforti cross (labelled E-O) show production of combinations of metabolites including mycotoxins which differ from those of the parental strains (labelled A and B).

FIG. 6 shows results of a real-time PCR assay to study induction of protease genes when P. roqueforti cultures of both parental isolates (labelled 88 and 92) and sexual offspring (labelled 118-127) were transferred to a milk casein inducing medium.

FIG. 7 illustrates a dose response ‘kill curve’ when spores of P. roqueforti strain 74-88 were exposed to a UV radiation source for between 0-150 seconds.

FIG. 8 illustrates some of the colour mutant strains of P. roqueforti produced after UV mutagenesis, including light brown, reddish-brown, green and intense blue colour sporulating mutants.

FIG. 9 shows the pigment development pathway of Aspergillus and Penicillium (taken from Krijgsheld et al., 2013).

FIG. 10 shows GC-MS “electronic nose” results for 5A, 12A, A22, B20.

FIG. 11 shows the lipolytic activity of the novel strains A22, B20, 5A and 12A.

FIG. 12 shows the proteolytic activity of the novel strains A22, B20, 5A and 12A.

FIG. 13 shows the growth rate of the novel strains A22, B20, 5A and 12A.

### DETAILED DESCRIPTION

### Sexual Crossing in Penicillium

Penicillium roqueforti

A worldwide strain collection of over 100 P. roqueforti isolates was established from various sources including cheese (for example blue cheeses), cheese ripening cultures, tortillas, bread, hay silage, contaminated foodstuffs and cooking oil, and samples from this collection were used in this study.

### Routine Culture Growth and Maintenance

Cultures of P. roqueforti were grown according to routine methods on potato dextrose agar (PDA—39 g potato dextrose agar power (Oxoid, UK) made up to 1 L with distilled water) or malt extract agar (MEA—20 g Malt Extract powder (Sigma, UK), 1 g Peptone (Oxoid, UK), 20 g Agar (Oxoid, UK) made up to 1 L with distilled water) on 9 cm Petri plates or as slope cultures in Universal tubes, using a swab to transfer mycelia from a pre-existing culture for inoculum as appropriate. Cultures were grown in the light for one week at 28° C., then sealed with Nescofilm and stored at 4° C. Long term stocks were also established under liquid nitrogen or as silica gel stock at 4° C.

### DNA Extraction

For extraction of genomic DNA from Penicillium species, mycelium was produced from liquid cultures. A spore solution was prepared by adding 1.5 ml 0.05% (v/v) tween 80 to a slope of a culture, which was agitated with a swab and recovered. The spore solution was then added directly to 250 ml conical flasks containing 50 ml of yeast extract glucose liquid growth medium (YEG: 8 g yeast extract, 40 g glucose/per litre). Cultures were grown at 28° C. on a rotary shaker at 150 rpm for 5 days. The resulting mycelium was collected by filtration through a double layer of Miracloth (Calbiochem, USA) and rinsed thoroughly with 0.1M potassium phosphate buffer (pH 7.0). The harvested mycelium was freeze-dried and stored at −80° C. until DNA extraction was performed. DNA was extracted using a DNeasy Plant Mini kit (Qiagen Ltd, UK) in accordance with the manufacturer's instructions. Concentrations of DNA were estimated by comparisons with lambda DNA quantity markers (Promega, USA) on 0.8% agarose gels (SeaKem® LE Agarose, Lonza Group Ltd, USA).

Screening of P. roqueforti for Mating-Type Genes

A multiplex PCR diagnostic test was employed to amplify mating-type genes and therefore determine the mating type of individual isolates. DNA was obtained from four isolates of P. roqueforti (74-9, 74-10, 74-41, and 74-42) which had previously been confirmed to be P. roqueforti, based on sequencing of the b-tubulin gene.

Sexual Assay of P. roqueforti

### Sexual Crosses

Various crosses were set up to study the potential for sexual reproduction of P. roqueforti. Six isolates of P. roqueforti used in cheese production comprising one MAT1-1 strains and five MAT1-2 strains were crossed in all possible pair wise combinations (n=5). In addition, crosses were set up between four isolates from field sources comprising two MAT1-1 and two MAT1-2 strains.

Spore suspensions of each isolate (5×10⁵ conidia ml⁻¹) were prepared as follows. Conidia were collected from two month old cultures grown at room temperature on slopes of MEA or PDA. A sterile 10 μl inoculating loop (Greiner) was gently rubbed across the surface of the sporulating colony on the slope. One loop full of conidia was transferred to 5 ml of a 0.05% (v/v) tween 80 solution containing 0.05% agar. The purpose of addition of the agar was to increase the viscosity of the medium in order to prevent the formation of stray colonies during subsequent inoculation. The resulting suspension was homogenized by using a vortex mixer and quantified using a Haemocytometer to estimate spore density and diluted to a target concentration of approximately 5×10⁵ conidia ml⁻¹. The spore suspension was then used immediately to avoid any change in viability due to storage.

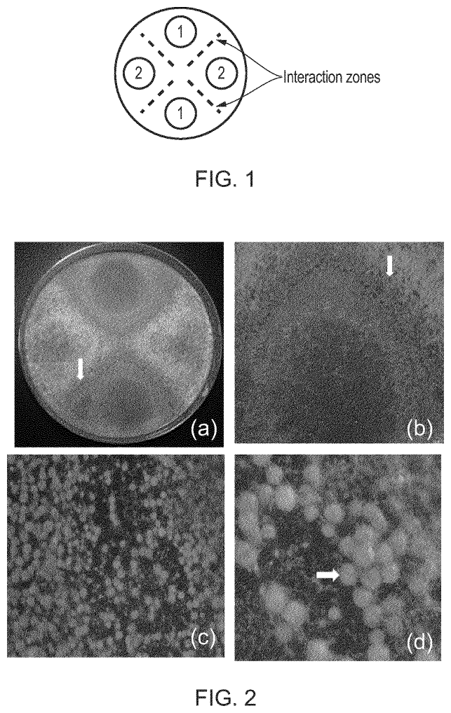

1 μl aliquots of each spore suspension was separately inoculated on to an oatmeal agar surface (Robert et al. 2007. CBS Yeasts Database, The Netherlands, Centraalbureau voor Schimmelcultures, Utrecht) ca. 4 cm apart and perpendicular to aliquots of conidia of the opposite mating type (O'Gorman et al 2009; Nature, 457, 471-4). This arrangement created four interaction or ‘barrage’ zones as colonies grew (FIG. 1). Petri dishes were sealed with one layer of Parafilm, incubated inverted in continuous darkness at 10° C. or 20° C., and examined periodically for cleistothecia over different lengths of time according to the particular experimental cross.

### Preparation of Single Ascospore Cultures

Mature cleistothecia from P. roqueforti cross 74-88×78-92 were removed and cleaned by rolling on agar and an ascospore suspension prepared by crushing of the cleistothecia.

More specifically, ascospore suspensions were prepared according to O'Gorman et al Nature, 457, 471-4. Mature cleistothecia were picked off from surrounding hyphae using a flame sterilised needle, observed under a Nikon-SMZ-2B dissecting microscope, taking care to avoid conidia where possible. 5-10 cleistothecia were transferred to a 4% (w/v) water agar plate and gently rolled across the agar surface using a sterilised needle tip to remove any adhering conidia, taking care to keep the cleistothecium intact. Next, 20-50 μl of pH 6 sterile Tween 80 (BDH)(0.05%) was pipetted into a sterile 1.5 ml Eppendorf tube and cleistothecia ruptured against the side of the tube in this small droplet, releasing a macerate of peridium, asci, ascospores and contaminating conidia. This was made up to a final volume of 500 μl in pH 6 sterile Tween 80 (0.05%) washing the cleistothecia contents from the side of the tube with final vortex-mixing for 1 min to release the ascospores. The concentration of the resulting ascospore suspension was determined using a haemocytometer and were diluted as appropriate.

Where appropriate, to inactivate any contaminating conidia, the suspension was then heat treated for different periods of time and at different temperatures to kill any remaining conidia whilst at the same time retaining viable ascospores e.g. heat treatment at 69° C. for 10 min (NB. in some species the ascospores and conidia were killed at the same temperature so this was not possible).

To isolate individual ascospores, 100 μl of a 5×10⁵ ascospore ml⁻¹ suspension (either with or without heat treatment) was spread inoculated on three defined areas of an ACM plate. Triplicate plates were prepared and incubated at 37° C. for 14 h. Single-spore cultures were established on ACM by transferring individual germinating ascospores with a LaRue lens cutter attached to a Nikon-Optiphot microscope or by picking up individual germinating ascospores with a flame sterilised platinum wire.

### Evidence of Recombination

The possible occurrence of recombination in the ascospore offspring of a sexual reproduction between P. roqueforti was assessed by examining the segregation of RAPD-PCR markers and the mating-type genotype in progeny. An initial screening of nine RAPD primers revealed five primers that were suitable for genotyping of P. roqueforti (OPAX16, UBC90, OPW10, OPAJ05, OPA11).

Results of Sexual Crosses in Penicillium roqueforti

The availability of the PCR-based MAT diagnostic tests allowed directed crosses to be set up between P. roqueforti isolates of known MAT1-1 or MAT1-2 identity. Results of crossing efforts are described below.

### Production of Cleistothecia

Crosses were set up involving five P. roqueforti isolates used in cheese manufacture, comprising one MAT1-1 isolate (74-88) crossed in all combinations to four MAT1-2 isolates (74-89, 74-90, 74-91 and 74-92) (Table 2). Crosses were incubated at 10° C., 15° C., and 20° C. to assess any capacity to undergo teleomorph stage development. Significantly, after four weeks incubation at all three temperatures, cleistothecia were observed and all were found to contain ascospores when squashed. Similar number of cleistothecia was produced at all three temperatures, however slightly higher numbers were apparent at 10° C. (Table 2). The crosses were then re-incubated for a further month and re-examined for the presence of cleistothecia. However, the longer incubation period yielded no apparent increase in the number of cleistothecia. The pairing of 74-88×78-90 produced the highest number of cleistothecia at all temperatures, and was very consistent with the formation of over 1,000 cleistothecia per plate (Table 2). Developing cleistothecia were initially white and soft when young, and then matured becoming pale orange-brown to bright yellow after 1-2 months of incubation. The cleistothecia formed along the barrage zones between isolates of opposite mating type (FIG. 2(a-d)).

Cleistothecia were also formed in some crosses between the four P. roqueforti isolates from field sources (Table 3).

TABLE 2
Number of cleistothecia produced by P. roqueforti crosses on oatmeal
agar medium at 10° C. in the dark after one month.

| | | Number of cleistothecia | | | |
|---|---|---|---|---|---|
| | | MAT1-2 | | | |
| Crosses | | 74-89 | 74-90 | 74-91 | 74-92 |
| MAT1-1 | 74-88 | > | >> | > | > |

Ratings indicate the mean number of cleistothecia produced from three replicate crosses on oatmeal agar in 9 cm diameter Petri dishes after incubating in the dark for 1 month at 10° C.: >more than 100 cleistothecia, >>more than 1,000 cleistothecia.

TABLE 3
Number of cleistothecia produced by P. roqueforti crosses
on oatmeal agar medium at 10° C. in the dark after one month.

| | | Number of cleistothecia |
|---|---|---|
| | | MAT1-2 |
| Crosses | | 74-4 |
| MAT1-1 | 74-84 | > |
| MAT1-1 | 74-86 | > |

Ratings indicate the mean number of cleistothecia produced from three replicate crosses on oatmeal agar in 9 cm diameter Petri dishes after incubating in the dark for 1 month at 10° C.: >more than 100 cleistothecia but less than 1,000.

### Evidence of Recombination

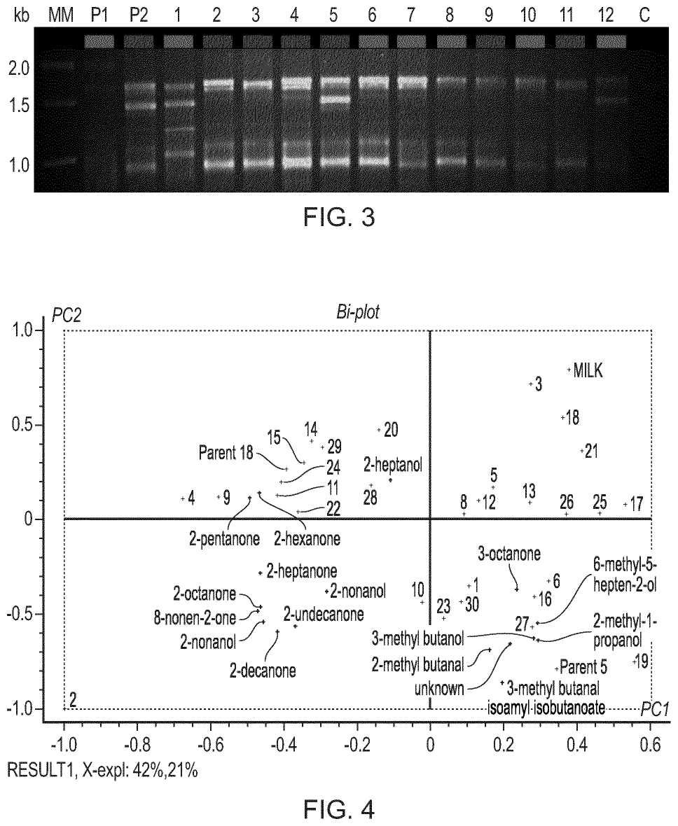

A recombination analysis of the ascospore offspring using molecular markers was conducted to confirm that meiosis had taken place. It was found that a heat shock of 70° C. for 10 min was sufficient to break the ascospore dormancy and activate germination, whilst killing any contaminating conidia. Distinct segregation patterns were clearly observed when six RAPD markers and the MAT genotype were scored in 12 ascospore progeny (FIG. 3 and Table 4). Unique genotypes were found in 83% of the progeny, with only two of the offspring identical to its parent (based on the markers examined (Table 4)). These results provide convincing data that P. roqueforti exhibits a heterothallic sexual breeding system.

TABLE 4
Genotypes^{a} in the parental isolates and 12 ascospore progeny
of a cross between P. roqueforti isolates 74-88 × 74-92

| | Mating | RAPD band^{b} | | | | | |
|---|---|---|---|---|---|---|---|
| Isolate | type | OPAJ05 | UBC 90 | OPAX 16 | OPW 10 | OPA 11 | Genotype^{c} |
| 74-88 | MAT1-1 | − | + | − | − | + | P1 |
| 74-92 | MAT1-2 | + | − | + | + | − | P2 |
| 88-92-1 | MAT1-1 | + | + | − | + | + | A |
| 88-92-2 | MAT1-2 | + | − | − | − | − | B |
| 88-92-3 | MAT1-2 | − | − | − | − | − | C |
| 88-92-4 | MAT1-2 | − | + | − | − | − | D |
| 88-92-5 | MAT1-2 | + | − | − | + | − | E |
| 88-92-6 | MAT1-1 | + | − | + | − | − | F |
| 88-92-7 | MAT1-1 | + | + | + | − | − | G |
| 88-92-8 | MAT1-1 | − | + | + | − | − | H |
| 88-92-9 | MAT1-2 | + | + | − | − | − | I |
| 88-92-11 | MAT1-1 | − | − | − | − | − | C |
| 88-92-13 | MAT1-2 | − | + | + | − | + | K |
| 88-92-18 | MAT1-1 | − | + | − | + | − | L |

| | | | | | |
|---|---|---|---|---|---|
| P- value(2- tailed)^{d} | 1.00 | 1.00 | 1.00 | 0.545 | 1.00 |

| | |
|---|---|
| Contingency χ^{ef} | 0.454(1) |

^{a}Genotypic characterization mating- type and RAPD-PCR bands.
^{b}RAPD-PCR bands amplified using operon primers OMT1 or R108. ‘+’ and ‘−’ denotes presence or absence, respectively, of particular amplicon.
^{c}The genotype of each progeny isolate, defined by unique combinations of mating-type and RAPD markers as distinct from the parental isolates (designated P1 and P2), is identified by a different letter of the alphabet
^{d}Fisher's exact test for deviation from the null hypothesis of independent assortment of mating-type and RAPD markers in the progeny (i.e. a 1:1:1:MAT1-1+:MAT1-1−:MATt1-2+:MAT1-2− ratio for each RAPD marker). Fisher's exact test was used instead of the χ² test because the expected frequencies were <5
^{e}To test for deviation from the null hypothesis of independent assortment of mating-type and RAPD markers in the progeny (i.e. an overall 1:1:1:1 MAT1-1+:MAT1-1−:MAT1-2+:MAT1-2−ratio for the sum of the RAPD markers).
^{f}Number in parenthesis indicates the degree of freedom.

The data above clearly demonstrates the ability of Penicillium roqueforti, used in the production of blue-veined cheeses, e.g. Roquefort, Danish blue, and Gorgonzola (Nichol, 2000) to sexually reproduce and produce progeny strains with different genotypes to either parent.

### Production of Cleistothecia

Crosses were set up involving five P. roqueforti isolates used in cheese manufacture, comprising one MAT1-1 isolate (74-88) crossed in all combinations to four MAT1-2 isolates (74-89, 74-90, 74-91 and 74-92) (Table 5). Crosses were incubated at 10° C., 15° C., and 20° C. to assess any capacity to undergo teleomorph stage development. Significantly, after four weeks incubation at all three temperatures, cleistothecia were observed and all were found to contain ascospores when squashed. Similar number of cleistothecia was produced at all three temperatures, however slightly higher numbers were apparent at 10° C. (Table 5). The crosses were then re-incubated for a further month and re-examined for the presence of cleistothecia. However, the longer incubation period yielded no apparent increase in the number of cleistothecia. The pairing of 74-88×78-90 produced the highest number of cleistothecia at all temperatures, and was very consistent with the formation of over 1,000 cleistothecia per plate (Table 5). Developing cleistothecia were initially white and soft when young, and then matured becoming pale orange-brown to bright yellow after 1-2 months of incubation. The cleistothecia formed along the barrage zones between isolates of opposite mating type (FIG. 2(a-d)).

Cleistothecia were also formed in some crosses between the four P. roqueforti isolates from field sources (Table 6). However, isolate 74-64 (MAT1-2) failed to form cleistothecia in any of the attempted crosses.

TABLE 5
Number of cleistothecia produced by P. roqueforti crosses on
oatmeal agar medium at 10° C. in the dark after one month.

| | | Number of cleistothecia | | | |
|---|---|---|---|---|---|
| | | MAT1-2 | | | |
| Crosses | | 74-89 | 74-90 | 74-91 | 74-92 |
| MAT1-1 | 74-88 | > | >> | > | > |

Ratings indicate the mean number of cleistothecia produced from three replicate crosses on oatmeal agar in 9 cm diameter Petri dishes after incubating in the dark for 1 month at 10° C.: >more than 100 cleistothecia, >>more than 1,000 cleistothecia.

TABLE 6
Number of cleistothecia produced by P. roqueforti crosses on
oatmeal agar medium at 10° C. in the dark after one month.

| | | Number of cleistothecia | |
|---|---|---|---|
| | | MAT1-2 | |
| Crosses | | 74-4 | 74-34 |
| MAT1-1 | 74-84 | > | — |
| MAT1-1 | 74-86 | > | — |

Ratings indicate the mean number of cleistothecia produced from three replicate crosses on oatmeal agar in 9 cm diameter Petri dishes after incubating in the dark for 1 month at 10 ° C.: —none, >more than 100 cleistothecia but less than 1,000.

### Proteolytic Activity, Lipolytic Activity and Growth Rate

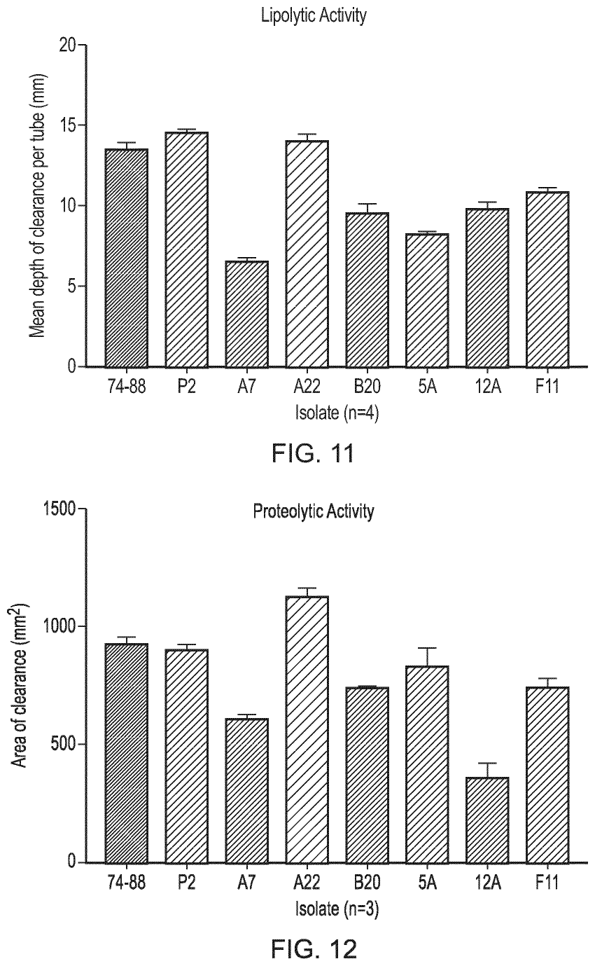

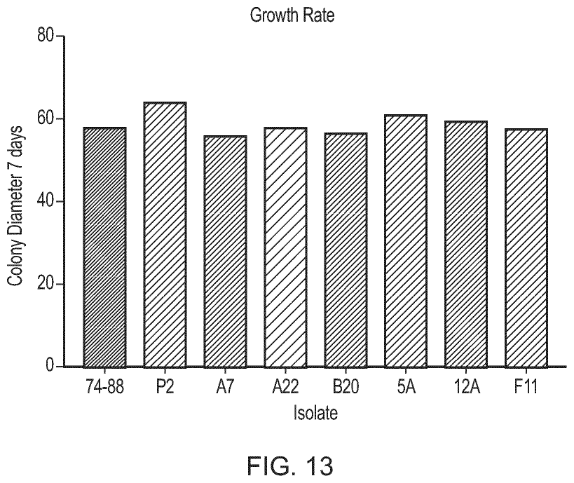

Table 7 below and FIGS. 11, 12 and 13 show the proteolytic activity, lipolytic activity and growth rate for the novel strains A22, B20, 5A and 12A. “Low” defined as below 25% percentile. “Med/Low” up to median. “Med/high” up to 75% percentile. “High” above 75% percentile. Penicillium roqueforti strains 74-88, P2, A7 and F11 may be found at the Nottingham University BDUN Collection.

TABLE 7
Proteolytic activity,
Lipolytic activity and Growth Rate of various strains

| | Proteolytic | Lipolytic | |
|---|---|---|---|
| Isolate | Activity | Activity | Growth Rate |
| 74-88 | Med/High | Med/High | Med/Low |
| P2 | Med/High | High | High |
| A7 | Low | Low | Low |
| A22 | High | Med/High | Med |
| B20 | Med/Low | Med/Low | Low |
| 5A | Med/High | Low | High |
| 12A | Low | Med/Low | Med/High |
| F11 | Med/Low | Med/High | Med/Low |

### Phenotypic Variations in the Properties of Penicillium Strains Produced by Sexual Crossing

### Differences in Flavour Properties

For example, a cross was set up between strains of Penicillium roqueforti derived from Roquefort and Gorgonzola type-blue cheeses. Thirty sexual progeny were recovered from this cross. The GC-MS technique was then used to determine levels of production of 16 volatiles linked to flavour production in an artificial cheese system based on growth in UHT milk in laboratory shake culture. Data for individual flavour volatiles were then pooled using principal component analysis (FIG. 4). This revealed that the parents (labelled Parent 5 and Parent 18 in bold font) occupied certain flavour spaces corresponding to the relative production of a variety of flavour volatiles. By contrast, the majority of the sexual offspring (labelled 1 to 30) occupied different flavour spaces on the principal component analysis, with some isolates (e.g. 2, 4, 9, 3, 18, 21) exhibiting quite different volatile flavour production from the parents, while others (e.g. 28, 8, 1) exhibited intermediate volatile flavour production between the two parents (FIG. 4). Thus, there is evidence of the production of different relative levels of flavour volatiles in the sexual offspring, likely to correlate with different flavour properties in cheese.

### Differences in Mycotoxin Production

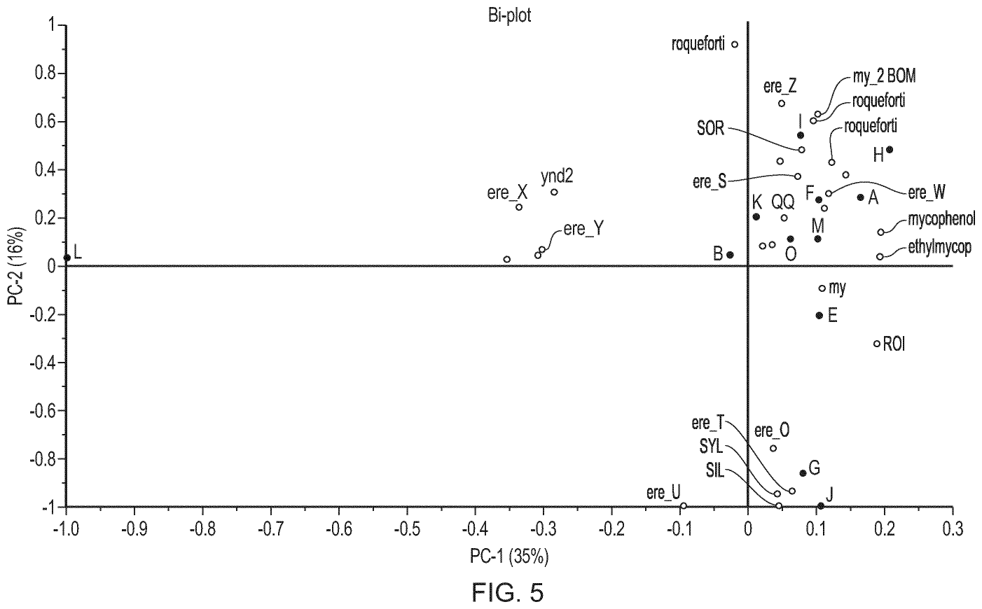

For example, a cross was set up between two blue cheese production strains of Penicillium roqueforti (A and B) and sexual progeny (E-O) were recovered from this cross. The HPLC technique was then used to determine levels of production of over 25 metabolites including known mycotoxins from representative offspring when grown in laboratory liquid culture media. Data for individual metabolites and mycotoxins were then pooled using principal component analysis (PCA) (FIG. 5). This revealed that the parents (labelled A and B in blue font) occupied certain areas of the PCA spaces corresponding to the relative production of the variety of metabolites including mycotoxins. The majority of the sexual offspring (also labelled in blue) exhibited intermediate metabolite and mycotoxin production between the two parents (e.g. K, O, M). However, some offspring showed quite different levels of metabolite including mycotoxin production (e.g. G, J, L). Thus, there is evidence of the production of different relative levels of metabolites including mycotoxins in the sexual offspring, offering the opportunity to select for offspring with lowered levels of mycotoxin production.

### Differences in Protease Profile

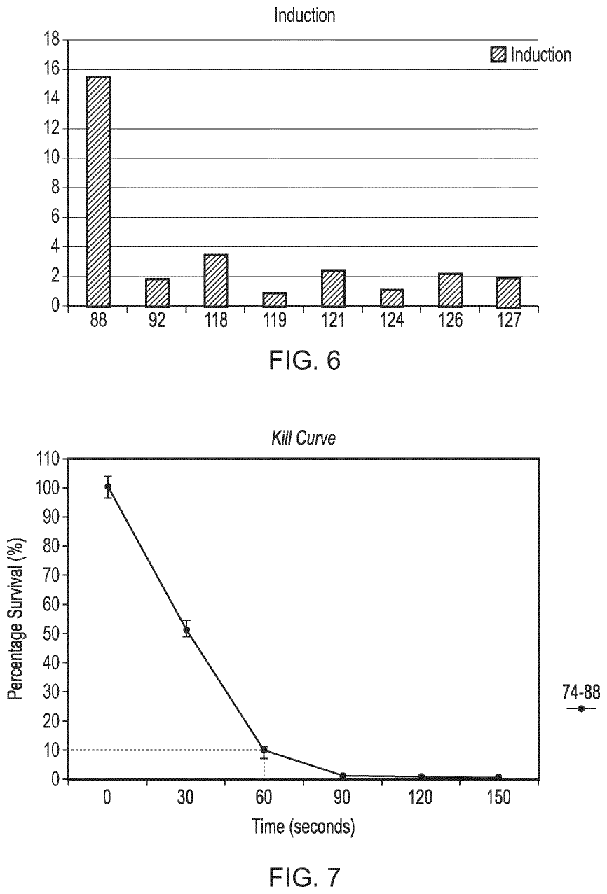

A cross was set up between two blue cheese production strains of Penicillium roqueforti (88 and 92) and six sexual progeny (118, 119, 121, 124, 126 and 127) were recovered from this cross. Both the parental isolates and the sexual offspring were then first grown on a non-milk based nutrient agar. They were then transferred to media containing milk protein (casein) and levels of induction of protease enzymes were determined using real-time PCR methodology, measuring induction of protease genes (FIG. 6). This analysis revealed differences in the protease profiles of the parental isolates (88 and 92), and that the sexual progeny exhibited levels of protease production which differed considerably from parent 88, but which were mostly similar to parent 92 although there were some more intermediate values (118) and some offspring showed protease production lower than either parent (119, 124) (FIG. 6). This small sample indicates that differences in protease production will be present in the sexual offspring relative to the parents, offering the opportunity to select for offspring with desirable levels of protease production.

### Mutagenesis of Penicillium Strains to Produce Different Coloured Spores

To produce mutants of Penicillium roqueforti by UV mutagenesis, a UV cabinet was switched on and allowed to warm up for 30 minutes (this also helps sterilize the UV box). A dose response curve was then performed to determine a suitable time to expose fungal material to result in a suitable decrease in viability. Fungal spores were produced on PDA and spore suspensions were made in sterile water aseptically and diluted to a concentration of 200,000 spores/ml, and cooled in a fridge for at least an hour. Spores were then transferred to a sterile 5 cm Petri dish and were continuously mixed using a sterilized magnetic stirrer in a 10 ml volume of spore suspension. The spores were then exposed to the UV radiation source at a certain distance from the lamp (standard between 30-40 cm below lamp) and aliquots of the spore suspension removed at suitable intervals (for example, between 30 seconds to 10 minutes) and stored in a fridge for 1 hour, using 3 technical replicates per time point. Spores were then transferred to 12 cm square plates containing PDA and then surviving spore numbers counted approximately 30 to 36 hours later.

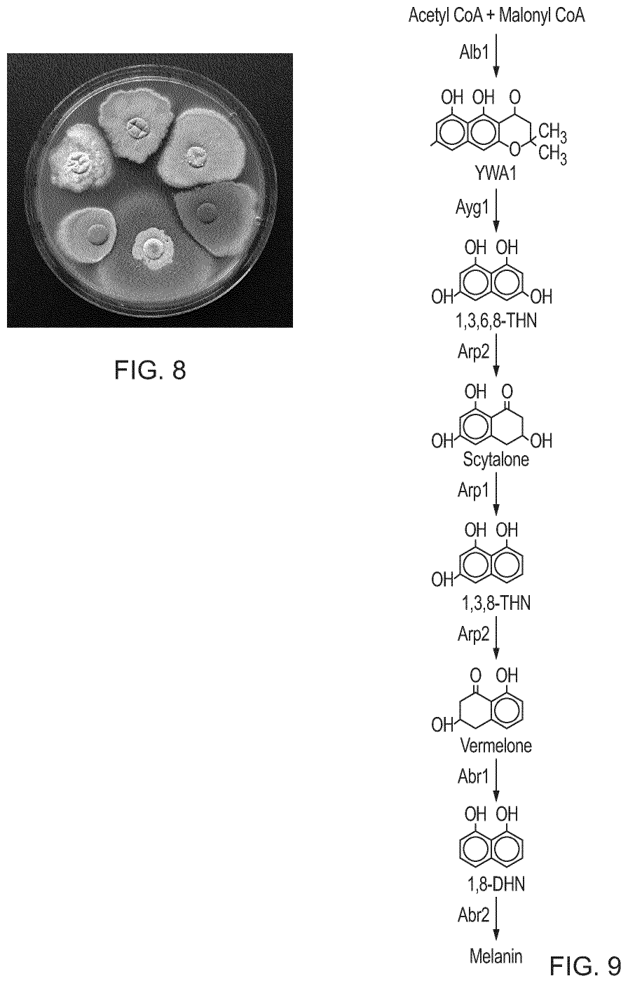

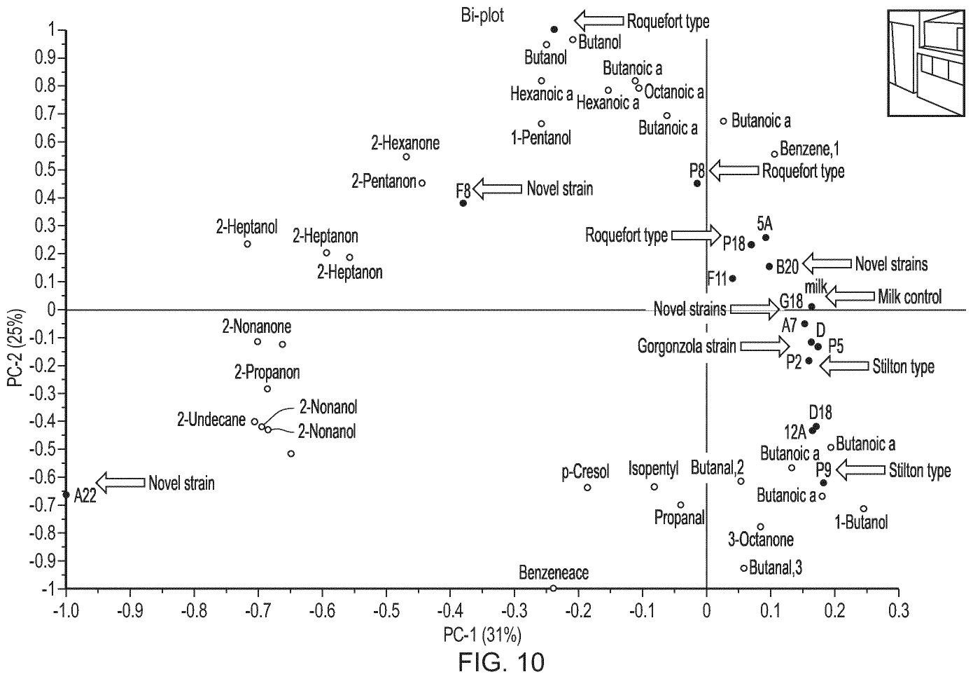

In the case of P. roqueforti strain 74-88, it was found that a suitable 5-10% survival rate resulted after exposure of spores for 60-90 seconds (FIG. 7). The resulting germinating fungal colonies that were exposed to UV radiation for 60-90 seconds were allowed to grow and sporulate. At this point mutants showing differences in colour from the wild type were then selected, such as light brown, reddish-brown, pink, green and intense blue mutants (FIG. 8).

### Production of a Blue Cheese Using a Penicillium Strain of the Invention.

In small-scale production trials, approximately 40 litres of whole, pasteurised milk was placed in hard plastic buckets. Milk was added at just over 34° C. to allow for the small volumes and the culture room was held at around 24° C. A mixed mesophilic starter culture from Christian Hansen and a gas giver, leuconostoc, from Danisco were added together with 5 ml of calcium chloride per bucket. Chymax rennet from Christian Hansen, 10 ml per bucket, diluted with 4 parts water was also added. The buckets were incubated, resulting in curd and whey formation. The curds were cut using an 11 mm square cutter and removed with a colander, then during moulding and turning further whey was removed by gravity, resulting in cheese wheels of perhaps 2.8-3 kg in size. The cheese was then turned a number of further times over 3-4 days. After one further day firming up is performed at around 16° C. followed by first and second salting in successive days then ripening at 12° C. Cheeses are then pierced during week 2 and by week 3 mould development should be apparent. Cheeses are then allowed to ripen for a suitable time between 2 and 3 months.

1. A method of producing strains of Penicillium by sexual reproduction comprising:
a. crossing complementary strains of Penicillium of mating type MAT1-1 and MAT1-2;
b. inoculating the isolates onto an agar medium;
c. incubating the inoculated isolates at a temperature of between about 10-20° C.; and
d. obtaining ascospores.

2. Method according to claim 1, further comprising the step of obtaining progeny strains from said ascospores.

3. Method according to claim 1 or 2, wherein said mating type strains are selected from any of Penicillium roqueforti strains 74-130; 74-144; 74-133; 74-146; strain Penicillium roqueforti 5A (74-256) deposited under the Budapest Treaty as CBS 145358, referred to herein as “5A”; strain Penicillium roqueforti 12A (74-257) deposited under the Budapest Treaty as CBS 145359, referred to herein as “12A”; strain Penicillium roqueforti A22 (74-160) deposited under the Budapest Treaty as CBS 145360, referred to herein as “A22”; strain Penicillium roqueforti B20 (74-170) deposited under the Budapest Treaty as CBS 145361, referred to herein as “B20”; a strain obtainable by sexually crossing strains of Penicillium roqueforti 74-130 and 74-144 and having similar characteristics to A22; a strain obtainable by sexually crossing strains of Penicillium roqueforti 74-133 and 74-146 and having similar characteristics to B20; variant strains having at least 99% sequence identity to any of the aforementioned strains.

4. Method according to claim 3, wherein strain 74-130 is sexually crossed with strain 74-144 and optionally produces strain A22 or a strain having similar characteristics to A22.

5. Method according to claim 3, wherein strain 74-133 is sexually crossed with strain 74-146 and optionally produces strain B20 or a strain having similar characteristics to B20.

6. Method according to any preceding claim, wherein said Penicillium is Penicillium roqueforti, Penicillium paneum, Penicillium glaucum or Penicillium camemberti.

7. Ascospores obtainable by a method according to any preceding claim and progeny strains obtainable from said ascospores.

8. A strain of Penicillium selected from:
a. strain Penicillium roqueforti 5A (74-256) deposited under the Budapest Treaty as CBS 145358, referred to herein as “5A”;
b. strain Penicillium roqueforti 12A (74-257) deposited under the Budapest Treaty as CBS 145359, referred to herein as “12A”;
c. strain Penicillium roqueforti A22 (74-160) deposited under the Budapest Treaty as CBS 145360, referred to herein as “A22”;
d. strain Penicillium roqueforti B20 (74-170) deposited under the Budapest Treaty as CBS 145361, referred to herein as “B20”;
e. a strain obtainable by sexually crossing strains of Penicillium roqueforti 74-130 and 74-144 and having similar characteristics to A22;
f. a strain obtainable by sexually crossing strains of Penicillium roqueforti 74-133 and 74-146 and having similar characteristics to B20;
g. variant strains having at least 99% sequence identity to the genome sequence of any of the strains defined in (a) to (f).

9. A derivative of a Penicillium strain having an altered pigment development pathway and capable of producing spores of a different colour from corresponding strains having an unaltered pigment development pathway.

10. A derivative strain according to claim 9, wherein said derivative is a derivative of ascospores or progeny strains according to claim 7 or a derivative of a strain according to claim 8.

11. A derivative strain according to claim 9 or 10, wherein the spores are any one or more of the following colours: light olive brown, light brown, olive brown, brown, reddish, reddish-brown, reddish-brown-pink, intense blue to light blue, green and albino.

12. A derivative strain according to any one of claims 9 to 11 having altered (increased or decreased or substantially eliminated) expression of one or more of the following metabolites: heptaketide naphthopyrone; 1,3,6,8-tetrahydroxynaphthalene; scytalone; 1,3,8-trihydroxynaphthalene; vermelone; 1,8-dihydroxynaphthalene, or metabolites produced as a result of the action of genes Alb1, Ayg1, Arp2, Arp1, Abr1, Abr2 from Penicillium species.

13. A derivative strain according to any of claims 9 to 12, wherein said pigment development pathway is altered by any one or more of: U.V., X-ray or chemical mutagenesis, gene editing, gene transformation, for example, to overexpress or downregulate one or more of the following genes: Alb1, Ayg1, Arp2, Arp1, Abr1, Abr2.

14. A strain according to claim 8, or a strain derived from ascospores according to claim 7, or a derivative according to any of claims 9 to 13, having altered protease and/or lipase levels and/or activity and/or altered levels of metabolites relative to either one or both parental strains used in the sexual cross, or relative to known strains used in the manufacture of dairy products.

15. A strain according to claim 8, or a strain derived from ascospores according to claim 7, or a derivative according to any of claims 9 to 13, when used in the production of a food product, produce food products having an altered organoleptic profile relative to either one or both parental strains used in the sexual cross, or relative to known strains used in the manufacture of dairy products.

16. A method for altering the colour of fungal spores, optionally spores of a Penicillium strain, comprising altering the pigment development pathway.

17. Fungal spores obtainable by a method according to claim 16, wherein said spores differ in colour from corresponding strains having an unmodified pigment development pathway.

18. A composition comprising a strain according to claim 8, or ascospores or a strain derived from ascospores according to claim 7, or a derivative according to any of claims 9 to 13, or fungal spores according to claim 17, and an acceptable carrier, such as an agriculturally acceptable carrier, a carrier suitable for nutraceutical administration, a carrier suitable for use in food applications, a carrier suitable for bioremediation applications.

19. Composition according to claim 18, comprising at least one additional bacteria or fungus.

20. Use of a composition according to claim 18 in any one or more of the following: (i) the production of or in a foodstuff, (ii) production of a nutraceutical, (iii) bioremediation, (iv) enzyme productions, (v) fermentation process, e.g. in the production of biofuels, (vi) in the production of or in animal feed.

21. Use according to claim 20, wherein said foodstuff is a cheese starter culture, cheese or a cheese product, such as a cheese sauce, cheese spread, dried cheese ingredient.

22. A cheese comprising a composition according to claim 18, a strain according to claim 8, or ascospores or a strain derived from ascospores according to claim 7, or a derivative according to any of claims 9 to 13, or fungal spores according to claim 17.

23. A cheese according to claim 22 which is a hard cheese or a soft cheese, a blue cheese, whether ripened by a Penicillium internal or external mould, optionally wherein the cheese is a Roquefort, Bleu de Bresse, Bleu du Vercors-Sassenage, Brebiblu, Cabrales, Cambozola (Blue Brie), Cashel Blue, Danish blue, blue Cheddar, Fourme d′Ambert, Fourme de Montbrison, Lanark Blue, Maytag Blue, Strathdon Blue, Blue Murder, Shropshire Blue, Dorset blue vinney, Brighton blue, Moyden's Wrekin blue, picos blue, cabrales, rokpol, dolcelatte, and Stilton, and some varieties of Bleu d′Auvergne, a Gorgonzola, a Brie, a Camembert or any other cheese ripened by a Penicillium internal or external mould.

24. A method of screening for strains having flavour profiles comparable to the strains of any preceding claim, comprising the steps of:
a. carrying out GC-MS analysis of strains B20, A22, 5A or 12A, variants or derivatives thereof as defined in any preceding claim;
b. carrying out GC-MS analysis of one or more query strains;
c. selecting those strains in (b) which group together with strains B20, A22, 5A or 12A, variants or derivatives thereof as shown in the GC-MS readings.

25. Strains obtainable by a screening method according to claim 24.
